(19) United States
(12) Patent Application Publication
(10) Pub. No.: US 2024/0008723 A1
(43) Pub. Date: Jan. 11, 2024

(54) WATER LINE AIR PURGE FOR AN ENDOSCOPE

(71) Applicant: Boston Scientific Scimed, Inc., Maple Grove, MN (US)

(72) Inventors: Ryan V. Wales, Northborough, MA (US); Paul Smith, Smithfield, RI (US); Jeff Gray, Sudbury, MA (US); Colby Harris, Norfolk, MA (US); Kurt Nicholas Robakiewicz, Upton, MA (US); Sean P. Fleury, Princeton, MA (US)

(73) Assignee: Boston Scientific Scimed, Inc., Maple Grove, MN (US)

(21) Appl. No.: 18/347,210

(22) Filed: Jul. 5, 2023

Related U.S. Application Data
(60) Provisional application No. 63/358,824, filed on Jul. 6, 2022.

(51) Int. Cl. A61B 1/015; A61B 1/00; A61B 1/12

(52) U.S. Cl. CPC A61B 1/015; A61B 1/00119; A61B 1/126

(57) ABSTRACT

Methods and systems for redirecting a flow of air/gas to flush fluid channels. An illustrative container and tube set for redirecting a flow of air may include a container, a water supply tube including a first lumen extending therethrough, a gas supply tube including a second lumen extending therethrough, a manifold including a first inlet for receiving air, a second inlet for receiving fluid from the container, a first air outlet, and a second fluid outlet, and a valve positioned within the manifold. When the valve is in a first configuration, the first inlet is in fluid communication with the first air outlet, and the second inlet is in fluid communication the second fluid outlet and when the valve is in a second configuration, the first inlet is in fluid communication with the second fluid outlet.

### CROSS-REFERENCE TO RELATED APPLICATIONS

This application claims the benefit of U.S. Prov. Pat. App. No. 63/358,824, filed Jul. 6, 2022, titled WATER LINE AIR PURGE FOR AN ENDOSCOPE, which is incorporated herein by reference.

### FIELD

This disclosure relates generally to medical fluid containers and tubing assemblies and methods, and particularly to a container and tube assembly to supply fluid and/or gas to an endoscope.

### BACKGROUND

Conventionally, endoscope devices have been widely used for performing diagnostic and/or therapeutic treatments. Once an endoscope procedure is complete, an initial cleaning of the endoscope is performed before the endoscope goes to be reprocessed. During this pre-cleaning process, water is flushed through the lens wash and irrigation working channels. After this, air is pushed through both channels to remove as much of the flushed water from the channels as possible. Currently, to achieve this, water flush followed by air flush is used to clear the irrigation and lens wash channels of debris. Alternatively, syringes may be manually filled and flushed down the various scope channels with water and room air to prepare the scope for complete reprocessing. These preparation steps may take time and are moderately labor intensive.

It is with these considerations in mind that the improvements of the present disclosure may be useful.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, an illustrative container and tube set arranged and configured to couple to an endoscope for use in an endoscopic procedure may comprise a container configured to contain a fluid, the container having a bottom portion and a top portion, a water supply tube including a first end, a second end, and a first lumen extending therethrough, wherein the first lumen is in selective fluid communication with the bottom portion of the container and the second end of the water supply tube is positioned external to the container, a gas supply tube including a first end, a second end, and a second lumen extending therethrough, wherein the second lumen is in operative fluid communication with the top portion of the container and the second end of the gas supply tube is positioned external to the container, a manifold including a first inlet for receiving air, a second inlet for receiving fluid from the container, a first air outlet, and a second fluid outlet, wherein the first air outlet is fluidly coupled to the second end of the gas supply tube and the second inlet is fluidly coupled to the second end of the water supply tube, and a valve positioned within the manifold, the valve movable between a first configuration and a second configuration to control an air flow path. When the valve is in the first configuration, the first inlet may be in fluid communication with the first air outlet, and the second inlet is in fluid communication the second fluid outlet when the valve is in the second configuration, the first inlet may be in fluid communication with the second fluid outlet.

Alternatively or additionally to any of the examples above, in another example, when the valve is in the first configuration air may be configured to flow from the first inlet to the first air outlet and into the container and fluid may be configured to flow from the container into the second inlet and to the second fluid outlet.

Alternatively or additionally to any of the examples above, in another example, when the valve is in the second configuration air may be configured to flow from the first inlet to the second fluid outlet.

Alternatively or additionally to any of the examples above, in another example, the second fluid outlet may be configured to be in fluid communication with one or more fluid channels of an endoscope.

Alternatively or additionally to any of the examples above, in another example, the container and tube set may further comprise a connecting channel extending between the gas supply tube and the water supply tube.

Alternatively or additionally to any of the examples above, in another example, the valve may be at least partially positioned in the connecting channel.

Alternatively or additionally to any of the examples above, in another example, the valve may comprise a rotating disc.

Alternatively or additionally to any of the examples above, in another example, in the first configuration, the rotating disc may be configured to fluidly isolate the gas supply tube and the water supply tube.

Alternatively or additionally to any of the examples above, in another example, in the second configuration, the rotating disc may be configured to block a flow of gas from the gas supply tube to the container and to block a flow of fluid from the container.

Alternatively or additionally to any of the examples above, in another example, the valve may comprise a slide valve.

Alternatively or additionally to any of the examples above, in another example, the slide valve may comprise a plurality of slide elements.

Alternatively or additionally to any of the examples above, in another example, the slide valve may comprise a first sliding element configured to selectively block a flow of gas from the gas supply tube to the container, a second sliding element configured to selectively block a flow of fluid from the container, and a third sliding element configured to selectively fluidly isolate the gas supply tube and the water supply tube.

Alternatively or additionally to any of the examples above, in another example, in the first configuration, the third sliding element may be disposed within a connecting channel extending between the gas supply tube and the water supply tube.

Alternatively or additionally to any of the examples above, in another example, in the second configuration, the first sliding element may be disposed within the gas supply tube and the second sliding element is disposed within the water supply tube.

In another example, a method of pre-cleaning an endoscope system may comprise flushing a fluid down a lens wash channel and an irrigation channel, actuating a valve to fluidly couple a gas supply line with a water supply line, and initiating a flow of gas through the lens wash channel and the irrigation channel.

In another example, a container and tube set arranged and configured to couple to an endoscope for use in an endoscopic procedure may comprise a container configured to contain a fluid, the container having a bottom portion and a top portion, a manifold, a lens wash supply tube including a first end coupled to the manifold, a second end, and a first lumen extending therethrough, wherein the first lumen is in fluid communication with the manifold and the second end of the lens wash supply tube is configured for connection to an endoscopic system, a first gas supply tube including a first end coupled to the manifold, a second end, and a second lumen extending therethrough, wherein the second lumen is in fluid communication with the manifold and the second end of the gas supply tube is configured for connection to an endoscopic system, a water supply tube including a first end, a second end coupled to the manifold, and a third lumen extending therethrough, wherein the third lumen is in fluid communication with the manifold and with the bottom portion of the container, a second gas supply tube including a first end, a second coupled to the manifold, and a fourth lumen extending therethrough, wherein the fourth lumen is in fluid communication with the manifold and with the top portion of the container, and a valve positioned within the manifold, the valve movable between a first configuration and a second configuration. When the valve is in the first configuration, the first lumen of the lens wash supply tube may be in fluid communication the third lumen of the water pickup tube, and the second lumen of the first gas supply tube may be in fluid communication with the fourth lumen of the second gas supply tube. When the valve is in the second configuration, the first lumen of the lens wash supply tube may be in fluid communication with the second lumen of the first gas supply tube.

Alternatively or additionally to any of the examples above, in another example, the container and tube set may further comprise a connecting channel extending between the second gas supply tube and the water supply tube.

Alternatively or additionally to any of the examples above, in another example, the valve may be at least partially positioned in the connecting channel.

Alternatively or additionally to any of the examples above, in another example, the valve may comprise a rotating disc.

Alternatively or additionally to any of the examples above, in another example, the valve may comprise a slide valve.

In another example, a container and tube set arranged and configured to couple to an endoscope for use in an endoscopic procedure may comprise a container configured to contain a fluid, the container having a bottom portion and a top portion, a cap coupled to the container adjacent the top portion thereof, a rotatable member disposed within the lid, a water supply tube including a first end coupled to the rotatable member, a second end, and a first lumen extending therethrough, wherein the first lumen is in selective fluid communication with the bottom portion of the container and the second end of the lens wash supply tube is positioned external to the container, a gas supply tube including a first end coupled to the rotatable member, a second end, and a second lumen extending therethrough, wherein the second lumen is in operative fluid communication with the top portion of the container and the second end of the gas supply tube is positioned external to the container, and a water pickup tube including a first end, a second end, and a third lumen extending therethrough, the first end in fluid communication with the bottom portion of the container and the second end positioned adjacent to the rotatable member.

Alternatively or additionally to any of the examples above, in another example, the rotatable member may be rotatable to selectively couple the third lumen of the water pickup tube with the first lumen of the lens wash supply tube.

Alternatively or additionally to any of the examples above, in another example, the rotatable member may be rotatable between a first configuration and a second configuration.

Alternatively or additionally to any of the examples above, in another example, when the rotatable member is in the first configuration, the third lumen of the water pickup tube may be in fluid communication with the first lumen of the lens wash supply tube.

Alternatively or additionally to any of the examples above, in another example, when the rotatable member is in the second configuration, the third lumen of the water pickup tube may be in fluid communication with the second lumen of the gas supply tube.

Alternatively or additionally to any of the examples above, in another example, the first end of the lens wash supply tube and the first end of the gas supply line may be coupled to the rotatable member.

Alternatively or additionally to any of the examples above, in another example, the rotatable member may comprise a rotatable disc.

Alternatively or additionally to any of the examples above, in another example, the rotatable member may comprise a rotatable gasket.

In another example, a container and tube set arranged and configured to couple to an endoscope for use in an endoscopic procedure may comprise a container configured to contain a fluid, the container having a bottom portion, an intermediate portion, and a top portion, an inflatable bladder having an interior disposed in the bottom portion of the container, a lens wash supply tube including a first end, a second end, and a first lumen extending therethrough, wherein the first lumen is in operative fluid communication with the intermediate portion of the container or with the interior of the inflatable bladder and the second end of the lens wash supply tube is positioned external to the container, and a gas supply tube including a first end, a second end, and a second lumen extending therethrough, wherein the second lumen is in fluid communication with the interior of the inflatable bladder and the second end of the gas supply tube is positioned external to the container.

Alternatively or additionally to any of the examples above, in another example, the inflatable bladder may be expandable to raise a level of the fluid in the container.

Alternatively or additionally to any of the examples above, in another example, the inflatable bladder may be deflatable to lower a level of the fluid in the container.

Alternatively or additionally to any of the examples above, in another example, the lens wash supply tube may be configured to axially translate within the container.

Alternatively or additionally to any of the examples above, in another example, the first end of the lens wash supply tube may comprise a needle.

Alternatively or additionally to any of the examples above, in another example, the first end of the lens wash supply tube may be configured to penetrate the inflatable bladder when the lens wash supply tube is axially advanced toward the bottom portion of the container such that the first lumen is in fluid communication with the interior of the inflatable bladder.

Alternatively or additionally to any of the examples above, in another example, the inflatable bladder may be self-sealing.

In another example, container and tube set arranged and configured to couple to an endoscope for use in an endoscopic procedure may comprise a container configured to contain a fluid, the container having a first chamber including a bottom portion and a top portion and a second chamber including a bottom portion and a top portion wherein the bottom portion of the second chamber is fluidly coupled to the bottom portion of the first chamber, a lens wash supply tube including a first end, a second end, and a first lumen extending therethrough, wherein the first lumen is in fluid communication with the bottom portion of the first chamber of the container and the second end of the lens wash supply tube is positioned external to the container, a gas supply tube including a first end, a second end, and a second lumen extending therethrough, wherein the second lumen is in fluid communication with top portion of the first chamber of the container and the second end of the gas supply tube is positioned external to the container, and a pressure control device fluidly coupled to the top portion of the second chamber of the container.

Alternatively or additionally to any of the examples above, in another example, the pressure control device may comprise a syringe.

Alternatively or additionally to any of the examples above, in another example, the pressure control device may comprise a pneumatic plunger.

Alternatively or additionally to any of the examples above, in another example, the pressure control device may comprise a solenoid.

Alternatively or additionally to any of the examples above, in another example, the pressure control device may be configured to raise a level of fluid in the first chamber by pushing fluid from the second chamber into the first chamber.

Alternatively or additionally to any of the examples above, in another example, the pressure control device may be configured to lower a level of fluid in the first chamber by drawing fluid from the first chamber into the second chamber.

In another example, a container and tube set arranged and configured to couple to an endoscope for use in an endoscopic procedure may comprise a container configured to contain a fluid, the container including a deformable bottom portion and a top portion, a lens wash supply tube including a first end, a second end, and a first lumen extending therethrough, wherein the first lumen is in fluid communication with the bottom portion of the container and the second end of the lens wash supply tube is positioned external to the container, and a gas supply tube including a first end, a second end, and a second lumen extending therethrough, wherein the second lumen is in fluid communication with top portion of the container and the second end of the gas supply tube is positioned external to the container. The bottom portion of the container may be configured to be compressed to raise a level of the fluid in the container and expanded to lower the level of the fluid in the container.

Alternatively or additionally to any of the examples above, in another example, the container and tube set may further comprising band disposed about the bottom portion of the container, the band configured to compress the bottom portion of the container.

Alternatively or additionally to any of the examples above, in another example, the band may be removable.

Alternatively or additionally to any of the examples above, in another example, in the absence of a biasing force on the bottom portion, the bottom portion of the container may expand to lower the level of the fluid in the container such that the first end of the lens wash supply tube is spaced a distance from the fluid in the container.

In another example, a container and tube set arranged and configured to couple to an endoscope for use in an endoscopic procedure may comprise a container having a first chamber configured to contain a fluid and including a bottom portion and a top portion and a second chamber fluidly isolated from the first chamber and including a bottom portion and a top portion, a cap coupled to the container adjacent a top end thereof, a rotatable member disposed within the cap, a lens wash supply tube including a first end coupled to the rotatable member, a second end, and a first lumen extending therethrough, wherein the first lumen is in selective fluid communication with the bottom portion of the first chamber of the container and the second end of the lens wash supply tube is positioned external to the container, a gas supply tube including a first end coupled to the rotatable member, a second end, and a second lumen extending therethrough, wherein the second lumen is in selective fluid communication with the top portion of the first chamber of the container and the second end of the gas supply tube is positioned external to the container, and a first water pickup tube including a first end, a second end, and a third lumen extending therethrough, the first end in fluid communication with the bottom portion of the first chamber of the container and the second end positioned adjacent to the rotatable member.

Alternatively or additionally to any of the examples above, in another example, the container and tube set may further comprising a second water pickup tube including a first end, a second end, and a fourth lumen extending therethrough, the first end in fluid communication with the bottom portion of the second chamber of the container and the second end positioned adjacent to the rotatable member.

Alternatively or additionally to any of the examples above, in another example, the rotatable member may be rotatable between a first configuration and a second configuration.

Alternatively or additionally to any of the examples above, in another example, when the rotatable member is in the first configuration, the third lumen of the first water pickup tube may be in fluid communication with the first lumen of the lens wash supply tube and the second lumen of the gas supply tube may be fluid communication with the top portion of the first chamber of the container.

Alternatively or additionally to any of the examples above, in another example, when the rotatable member is in the second configuration, the first lumen of the lens wash supply tube maybe in fluid communication with the second chamber and the second lumen of the gas supply tube may be fluid communication with the second chamber.

Alternatively or additionally to any of the examples above, in another example, the rotatable member may comprise a rotatable disc.

Alternatively or additionally to any of the examples above, in another example, the rotatable member may comprise a rotatable gasket.

In another example, a cap configured to couple to a distal end of an endoscope for use in an endoscopic procedure may comprise a tubular body extending from a first end to a second end and defining a tubular sidewall, an end wall disposed adjacent to the second end of the tubular body, the end wall extending generally orthogonal to a longitudinal axis of the tubular body, and a securement mechanism disposed on an inner surface of the tubular sidewall adjacent the first end of the tubular body. The cap may be configured to be releasably coupled to a distal end of an endoscope.

Alternatively or additionally to any of the examples above, in another example, when the cap is releasably coupled to the endoscope, the end wall may be distally spaced from a distal end surface of the endoscope.

Alternatively or additionally to any of the examples above, in another example, the securement mechanism may comprise a plurality of helical threads.

Alternatively or additionally to any of the examples above, in another example, the securement mechanism may comprise a detent configured to mate with a corresponding groove on the endoscope.

Alternatively or additionally to any of the examples above, in another example, the securement mechanism may comprise a groove configured to mate with a corresponding detent on the endoscope.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3A depicts an endoscope system with endoscope, light source, water reservoir, and tubing assembly for hybrid air, lens wash and irrigation fluid delivery, wherein the system is activated to deliver air to atmosphere;

FIG. 3B depicts the endoscope system of FIG. 3A, wherein the system is activated to deliver air to a patient through the patient end of the endoscope;

FIG. 3C depicts the endoscope system of FIG. 3A, wherein the system is activated to deliver lens wash fluid through the patient end of the endoscope;

FIG. 3D depicts the endoscope system of FIG. 3A, wherein the system is activated to deliver irrigation fluid through the patient end of the endoscope;

FIG. 4 depicts a hybrid endoscope system including a video processing unit, connector portion, peristaltic irrigation pump, water reservoir and top, coaxial gas and lens wash supply tubing, upstream and downstream irrigation supply tubing, and alternative gas supply tubing;

FIG. 5 depicts a schematic cross-sectional view of a tubing assembly suitable for use with an endoscope system, with a tubing manifold, a reservoir, and tubing, according to an embodiment of the present disclosure

FIG. 6A depicts an illustrative valve assembly for controlling a flow of fluid suitable for use with an endoscope system in a first configuration, according to an embodiment of the present disclosure;

FIG. 6B depicts the illustrative valve assembly of FIG. 6A in a second configuration, according to an embodiment of the present disclosure;

FIG. 7A depicts another illustrative valve assembly for controlling a flow of fluid suitable for use with an endoscope system in a first configuration, according to an embodiment of the present disclosure;

FIG. 7B depicts the illustrative valve of FIG. 7A in a second configuration, according to an embodiment of the present disclosure;

FIG. 8A depicts an illustrative container and tube assembly for use with an endoscope system in a first configuration, according to an embodiment of the present disclosure;

FIG. 8B depicts the illustrative container and tube assembly of FIG. 8A in a second configuration, according to an embodiment of the present disclosure;

FIG. 9A depicts another illustrative container and tube assembly for use with an endoscope system in a first configuration, according to an embodiment of the present disclosure;

FIG. 9B depicts the illustrative container and tube assembly of FIG. 9A in a second configuration, according to an embodiment of the present disclosure;

FIG. 10 depicts another illustrative container and tube assembly for use with an endoscope system, according to an embodiment of the present disclosure;

FIG. 11A depicts another illustrative container and tube assembly for use with an endoscope system in a first configuration, according to an embodiment of the present disclosure;

FIG. 11B depicts the illustrative container and tube assembly of FIG. 11A in a second configuration, according to an embodiment of the present disclosure;

FIG. 12A depicts another illustrative container and tube assembly for use with an endoscope system in a first configuration, according to an embodiment of the present disclosure;

FIG. 12B depicts the illustrative container and tube assembly of FIG. 12A in a second configuration, according to an embodiment of the present disclosure;

FIG. 13A depicts another illustrative container and tube assembly for use with an endoscope system in a first configuration, according to an embodiment of the present disclosure;

FIG. 13B depicts the illustrative container and tube assembly of FIG. 13A in a second configuration, according to an embodiment of the present disclosure;

FIG. 13C depicts the illustrative container and tube assembly of FIG. 13A in an alternative second configuration, according to an embodiment of the present disclosure;

FIG. 14A depicts another illustrative container and tube assembly for use with an endoscope system in a first configuration, according to an embodiment of the present disclosure;

FIG. 14B depicts the illustrative container and tube assembly of FIG. 14A in a second configuration, according to an embodiment of the present disclosure;

FIG. 15A depicts another illustrative container and tube assembly for use with an endoscope system in a first configuration, according to an embodiment of the present disclosure;

FIG. 15B depicts the illustrative container and tube assembly of FIG. 1A in a second configuration, according to an embodiment of the present disclosure; and

FIG. 16 depicts an illustrative cap for use with an endoscope system, according to an embodiment of the present disclosure.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes description of a manifold, bottle and tube set, and/or cap suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. Once an endoscope procedure is complete, an initial cleaning of the endoscope is performed before the endoscope goes to be reprocessed. During this pre-cleaning process, water is flushed through the lens wash and irrigation working channels. After this, air is pushed through both channels to remove as much of the flushed water from the channels as possible. Currently, to achieve this, water flush followed by air flush is used to clear the irrigation and lens wash channels of debris. Alternatively, syringes may be manually filled and flushed down the various scope channels with water and room air to prepare the scope for complete reprocessing. These preparation steps may take time and are moderately labor intensive. Disclosed herein are methods and systems to pre-clean the lens wash and irrigation lines within the endoscope.

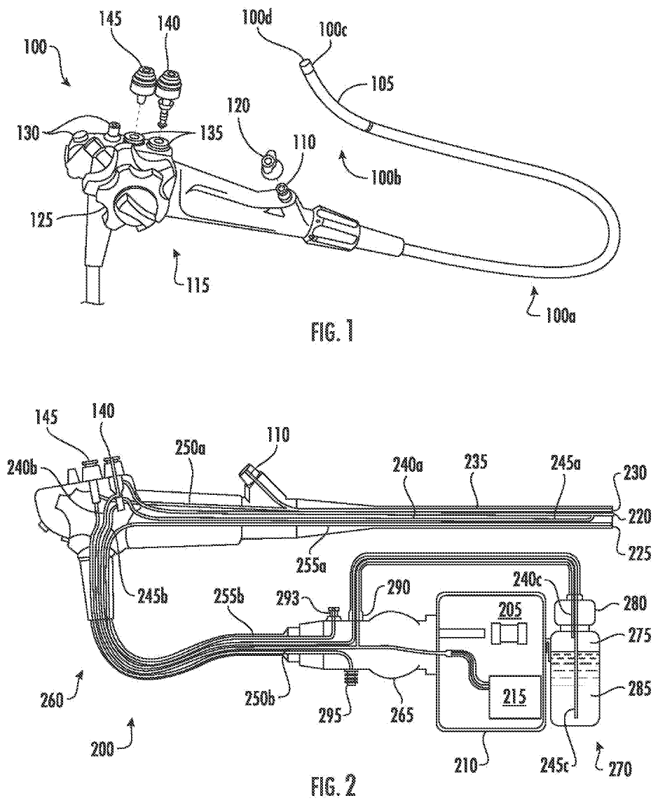

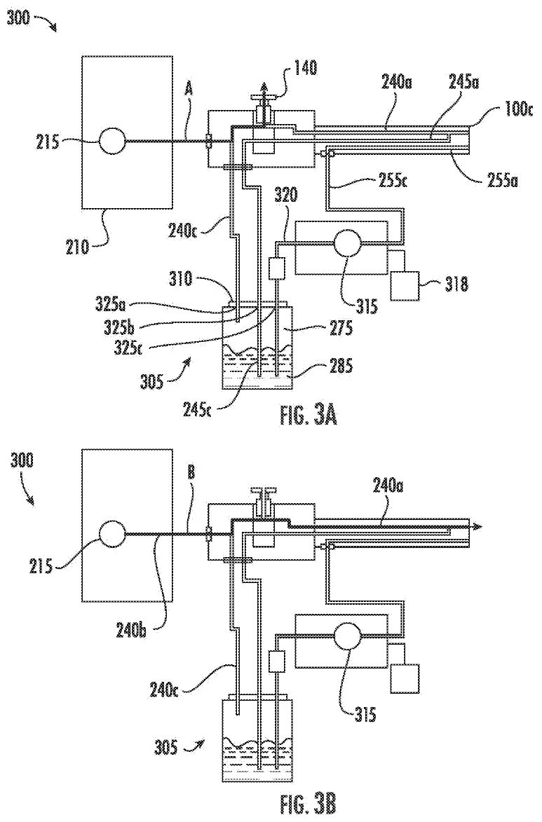

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connection portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

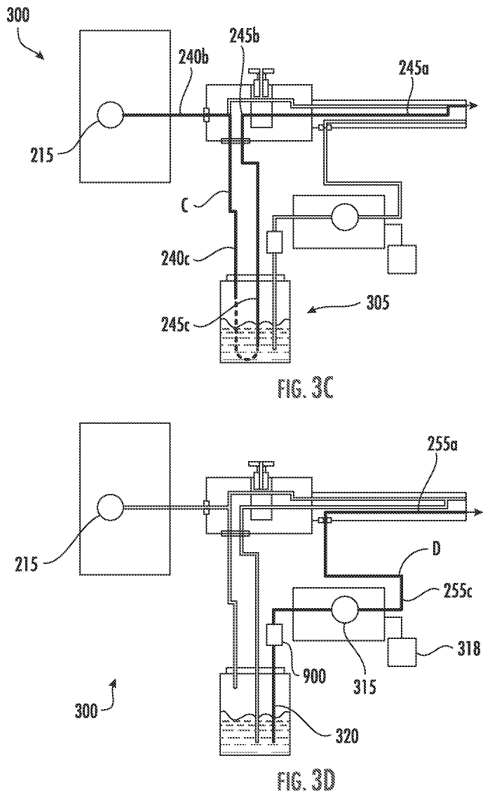

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream irrigation tubing 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 240c, upstream irrigation supply tubing 320, and/or downstream irrigation supply tubing 255c to help prevent backflow of water from either or both of the lens wash and irrigation tubing in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 240c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash 240c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 240c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 240c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope, and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to delivery water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

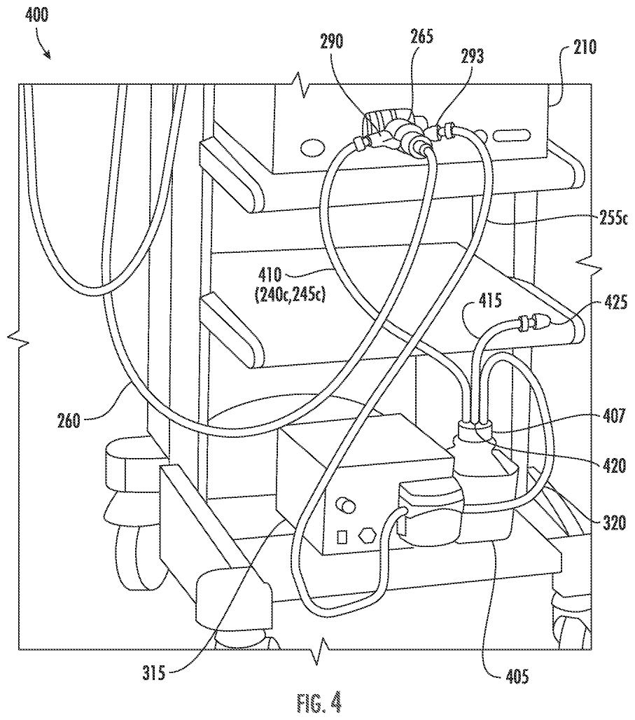

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, respectively, and alternative gas (e.g., CO₂) supply tubing 415. A length of the alternative gas supply tubing 415 passes from one end positioned in the gas gap 275 (see FIG. 2) between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. Generally, the flow of CO₂ through the endoscope 100 is similar to the flow of air. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivery gas through alternative gas (e.g., CO₂) supply tubing 415. The irrigation function may be accomplished in a similar manner as the operation described above with respect to FIG. 3D.

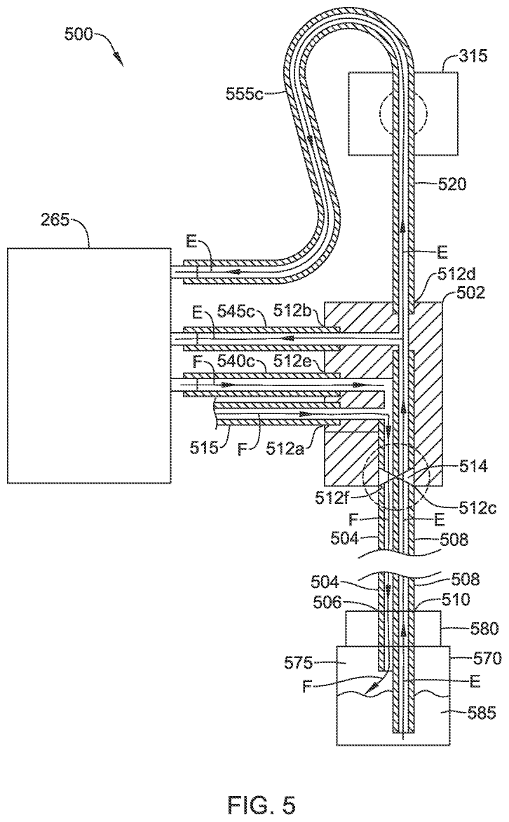

As described above, once an endoscope procedure is complete, an initial pre-cleaning of the endoscope is performed before the endoscope goes to be reprocessed. During this pre-cleaning process, water is flushed through the lens wash and irrigation working channels. After this, air is pushed through both lens wash and irrigation supply lines 245a, 255a to remove as much of the flushed water from the channels 245a, 255a as possible. FIG. 5 depicts a schematic cross-sectional view of an illustrative tubing assembly 500, arranged and configured for distributing fluid to an endoscope system. The tubing assembly 500 may be further arranged and configured to divert air or CO₂ supplied by the air pump 215 (or other gas source) to the lens wash supply line 245a and the irrigation supply line 255a to perform the pre-cleaning process. The system apart from the tubing assembly includes components similar to the endoscope and endoscope systems described with regard to FIGS. 1-4; however, not all features may be described or shown here if not pertinent to the fluid circuit of the system.

The fluid reservoir 570 is shown with reservoir top or cap 580, which may be removably attachable to the top portion of the reservoir 570 (e.g., in a bottle and threaded cap arrangement). The cap 580 may be removably attached in order to replenish fluid in the reservoir when it becomes depleted. Alternatively, the reservoir bottom 570 and top 580 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

The reservoir top 580 may be connected in fluid communication with a tubing manifold 502 via a shared gas supply/alternate gas supply tubing (or gas supply tubing) 504. The shared tubing 504 extends from a second end at an opening of the manifold 502 (external to the reservoir 570) through a reservoir opening 506 in the reservoir top 580, terminating at a first end within the reservoir gap 575, at or below the top opening 506, but not extending into the remaining fluid 585 in the reservoir 570. A lumen extends through the gas supply tubing 504 for receiving a flow of air and/or gas therethrough. The lumen of the gas supply tubing 504 is in operative fluid communication with the top portion of the reservoir 570. The reservoir top 580 may also be connected in fluid communication with tubing manifold 502 via a portion of a shared lens wash supply/irrigation supply tubing (or water supply tubing) 508. The water supply tubing 508 extends from a second end at an opening of manifold 502 (external to the reservoir 570) through a reservoir opening 510 in the top 580, terminating in a first end within the remaining fluid 585 at or substantially at the bottom of reservoir 570. A lumen extends through the water supply tubing 508 for receiving a flow of fluid therethrough. The lumen of the lens wash supply/irrigation supply tubing 508 is in selective operative fluid communication with the bottom portion of the reservoir 570. In some embodiments, the gas supply tubing 504 and the water supply tubing 508 may enter the top 580 through a single or common opening. For example, the gas supply tubing 504 and the water supply tubing 508 may be coaxially arranged. Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference.

The tubing assembly 500 may also include an alternate gas supply tubing 515 extending from an alternate gas source (not explicitly shown) to an opening of the tubing manifold 502. The alternate gas supply tubing 515 may include a fitting (not explicitly shown) at a gas source end of the portion of tubing 515. The fitting may be one or more of a valve, an adaptor, a filter, etc., for one or more of a variety of uses, such as filtering particulate in the fluid, closing off flow through the tubing, preventing flow through the tubing in a certain direction, adapting to a source fitting, etc., and the like.

A portion of a gas supply tubing 540c and a portion of lens wash supply tubing 545c may extend from an opening of the manifold 502, respectively, and may be connected in fluid communication with the endoscope at gas/lens wash connection on the connector portion 265 of the umbilical. The gas supply tubing 540c is connected in fluid communication with a gas pump (not explicitly shown) and gas feed line (not explicitly shown), and the lens wash supply tubing 545c is connected in fluid communication with lens wash feed line (not explicitly shown), within connector portion 265. The gas supply tubing 540c is in fluid communication through tubing manifold 502 with the gas supply tubing 504. Similarly, the lens wash supply tubing 545c is in fluid communication through tubing manifold 502 with the shared lens wash/irrigation (or water) supply tubing 508.

A portion of upstream irrigation supply tubing 520 extending from an opening of manifold 502 is in fluid communication with a portion of downstream irrigation supply tubing 555c. The portion of irrigation supply tubing 520 is upstream of an inlet to pump 315 and is in fluid communication through manifold 502 with the shared lens wash supply and irrigation supply tubing 508. The portion of irrigation supply tubing 555c is downstream of an outlet to pump 315 and is connected in fluid communication with the endoscope at the irrigation connection on the connector portion 265 of the umbilicus. The portion of irrigation supply tubing 555c is connected in fluid communication with irrigation feed line (not explicitly shown), within connector portion 265.

The tubing manifold 502, reservoir cap 580 and various portions of supply tubing may be connected in fluid communication between an alternate gas source (not shown) and the manifold 502, between the manifold 502 and the fluid reservoir 570, and between the manifold 502 and the video processing unit (not explicitly shown) of the endoscope. The manifold 502 may have a number of openings 512a, 512b, 512c, 512d, 512e, 512f (collectively, 512), arranged and configured to receive the manifold ends of the gas supply tubing 540c, upstream irrigation supply tubing 520, lens wash supply tubing 545c, and alternate gas supply tubing 515, as well as the manifold (or second) ends of the shared lens wash/irrigation supply tubing 508 and the shared gas supply/alternate gas supply tubing 204.

In embodiments, the reservoir top 580 may be a threaded cap removably attached to the remaining portion of reservoir 570. The cap may be arranged and configured to be compatible with a single type of reservoir (e.g., bottle type), or compatible with different types of reservoirs (e.g., universal threading to accept different bottle types). In removable embodiments, top 580 may include reservoir gasket (not explicitly shown) to help ensure a pressure-tight seal within the reservoir, when attached.

The shared lens wash/irrigation supply tubing 508 extends through the reservoir opening 510 to the second end of the tubing connected in fluid communication within lens wash/irrigation opening 512c in the manifold 502. The shared gas/alternate gas supply tubing 504 extends through reservoir opening 506 to the manifold end of the tubing connected in fluid communication within gas/alternate gas opening 512f in the manifold 502. As shown, the gas supply tubing 540c may extend from the connector portion 265 to the manifold end of the tubing connected in fluid communication within gas/alternate gas opening 512e in the manifold 502, the lens wash supply tubing 545c extends from the connector portion 265 to the manifold end of the tubing connected in fluid communication within lens wash opening 512b in the manifold 502, the downstream portion of irrigation tubing 555c extends from the connector portion 265 to the outlet of the irrigation pump 315 and the upstream portion of irrigation tubing 520 extends from the inlet of the pump to the manifold end of the tubing 520 connected in fluid communication within the irrigation opening 512d in the manifold 502, and the portion of alternate gas supply tubing 515 extends from the alternate gas source (not explicitly shown) to the manifold end of the tubing connected in fluid communication within alternate gas opening 512a in the manifold 502.

Certain of the openings 512 in the manifold 502 may be fluidly connected through channels in the manifold with other of the openings 512 in the manifold to establish flow paths for the gas supply, alternate gas supply, irrigation supply and/or lens wash supply. For example, as shown in FIG. 5, openings 512a, 512e, and 512f are fluidly connected to each other through a common channel within manifold 502 (e.g., flow path F). Further, as shown in FIG. 5, openings 512b, 512c, and 512d are fluidly connected to each other through a common channel in manifold 502 (e.g., flow path E).

In embodiments, the use of tubing assembly 500, with an endoscope during a procedure with a fluid circuit having the same or similar tubing as system 500, may follow flow paths E and F depicted in FIG. 5. For example, with respect to flow path F, when the air pump 215 is activated and gas/lens wash valve 140 is depressed, air flows from video processing unit 210, along the portion of gas supply tubing 540c through opening 502e of the manifold, exits the manifold through opening 512f and flows along the shared gas/alternate gas supply tubing 504 through the opening 506 in the cap 580 and into the gap 575 in fluid reservoir 570. Flow from alternate gas source through tubing 515 and opening 512a is closed off, allowing the gas supply to pressurize the fluid reservoir. Further, with respect to flow path E, when the gas supply pressurizes the fluid reservoir, fluid is forced along the shared lens wash/irrigation supply tubing 508, through the opening 510 in the cap 580, and enters the manifold through opening 512c. In the absence of the irrigation pump 315 being activated or the irrigation supply tubing not being used or closed off (e.g., a seal or membrane, fitting or valve, as described below), fluid continues to flow as lens wash fluid, exiting opening 512b and flowing along tubing 545c to the connector portion 265 of umbilical 260 connected to video processing unit 210. In the event a portion of irrigation supply tubing 520 is connected to manifold 502 and is open to flow when irrigation pump 315 is activated, fluid is drawn from the fluid reservoir 570 along the same path as lens wash fluid into the manifold 502, at which point it branches off from the lens wash fluid path and exits through opening 512d, along the upstream portion of irrigation supply tubing 520, and is pumped through the downstream portion of irrigation supply tubing 555c to the connector portion 265 of umbilical 260 connected to video processing unit 210. Fluid for irrigation may be drawn by the pump 315 from the fluid reservoir at the same time as fluid for lens wash is pushed to the endoscope by the gas supply, or the functions may be performed separately.

The tubing assembly 500 may further include an actuatable valve 514 positioned within the manifold 502. Alternatively, the valve 514 may be provided external to the manifold 502 and/or as a separate manifold fluidly coupled to the openings 512c, 512f The valve 514 may be used to divert or control an air flow or alternative gas flow supplied by the air pump 215 (or other gas source) to the lens wash supply line 245a and the irrigation supply line 255a within the endoscope. Generally, the valve 514 may be configured to selectively fluidly couple the gas supply tubing 504 with the water supply tubing 508. This selective fluid coupling may allow pressurized air/gas to flow through the lens wash tubing 545c and/or the irrigation tubing 520, 555c to the lens wash supply line 245a and the irrigation supply line 255a within the endoscope.

The valve 514 may be any valve that may be actuated to control a flow of fluid. For example, the valve 514 may be an active valve that is operated in a binary manner as an on/off valve or switch to allow flow to be turned on or allow flow to be turned off. Some illustrative valve types may include, but are not limited, stop cock valves, solenoid valves, slide valves, etc.

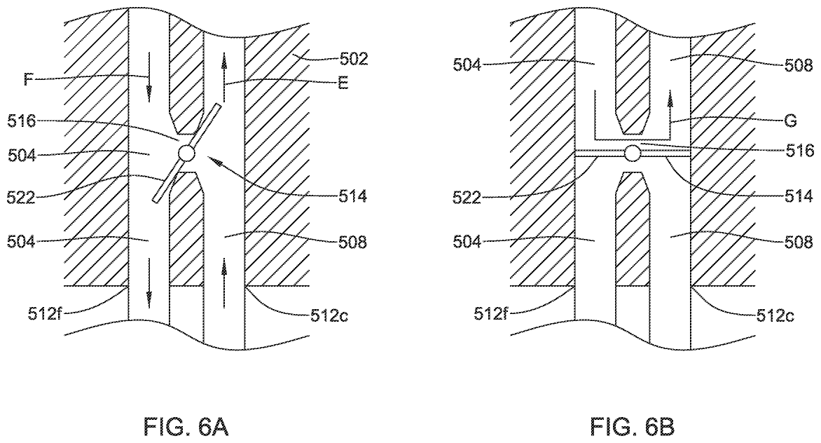

FIG. 6A is a schematic cross-sectional view of the valve 514 shown in the dotted border of FIG. 5, in a first configuration and FIG. 6B is a schematic cross-sectional view of the valve 514 shown in the dotted border of FIG. 5, in a second configuration. The valve 514 is positioned at least partially within a connecting channel 516 extending between the gas supply tubing 504 and the water supply tubing 508. The connecting channel 516 fluidly couples the gas supply tubing 504 and the water supply tubing 508. In the illustrative embodiment of FIGS. 6A and 6B, the valve 514 includes a rotating or pivoting disc 522. The valve disc 522 may have a size and shape similar to the cross section of the tube assembly at the connecting channel 516 such that the valve disc may provide a fluid tight seal at the connecting channel 516. Other valve types and valve configurations may be used, as desired. While not explicitly shown, an actuation mechanism, such as, but not limited to a handle or wheel, may be positioned exterior to the manifold 502 such that the user may selectively move the valve 514 between the first and second configurations.

In the first configuration illustrated in FIG. 6A, the valve 514 is positioned to allow for normal function of the endoscope. For example, when the valve 514 is in the first configuration, the valve disc 522 contacts an inner surface of the gas supply tubing 504 and an inner surface of the water supply tubing 508 to fluidly seal the connecting channel 516. The valve disc 522 fluidly isolates the gas supply tubing 504 and the water supply tubing 508 such that the gas inlet 512a, 512e is in fluid communication with the air outlet 512f and the water inlet 512c is in fluid communication the second water outlet 512b, 512d. The manifold 502 directs air or gas from the processing unit 210 (or other external source) into the water reservoir 570, along flow path F. The flow of air/gas can be controlled at the gas/water valve 140, as described above. For example, when the gas/water valve 140 is depressed, the air/gas is received into the manifold 502 at a first inlet or opening 512a, 512e and exits the manifold 502 at an air outlet opening 512f via the gas supply tubing 504. Pressure generated by the air within the sealed water reservoir 570 directs water up the water supply tubing 508. Water enters the manifold at a water inlet 512c via the water supply tubing 508. Depending on whether or not the irrigation pump 315 is active, water travels along flow path E (a) to the lens wash supply line 245a or (b) to both the lens wash supply line 245a and the irrigation supply line 255a.

When the endoscopic procedure is complete and pre-cleaning of the endoscope 100 is to be performed, the valve 514 is actuated to its second configuration, as shown in FIG. 6B. This may be performed by manually turning an actuation mechanism or by activating an electronically controlled actuator. When the valve 514 is in the second configuration, the gas inlet 512a, 512e is in fluid communication with the water outlet 512b, 512d via the connecting channel 516. For example, the valve disc 522 is rotated or pivoted to block the flow of air/gas within the gas supply tubing 504 and the flow of water from the reservoir 570 within the water supply tubing 508. The valve 514 may be located such that when the valve 514 is in the second configuration, the lumen of the gas supply tubing 504 is in fluid communication with the lumen of the water supply tubing 508 via the connecting channel 516. Further, in the second configuration, the valve disc 522 prevents air/gas from entering the reservoir 570 and deviates the air/gas into the water supply tubing 508, as shown at flow path G. The air then enters the endoscope and pushes water out of the irrigation and lens wash channels 255a, 245a.

To perform the pre-cleaning procedure, water may first be flushed through the lens wash and irrigation channels 245a, 255a with the valve 514 in the first configuration. The valve 514 may then be actuated to the second configuration and air/gas is directed into the lens wash and irrigation channels 245a, 255a to remove as much of the flushed water from the channels 245a, 255a as possible.

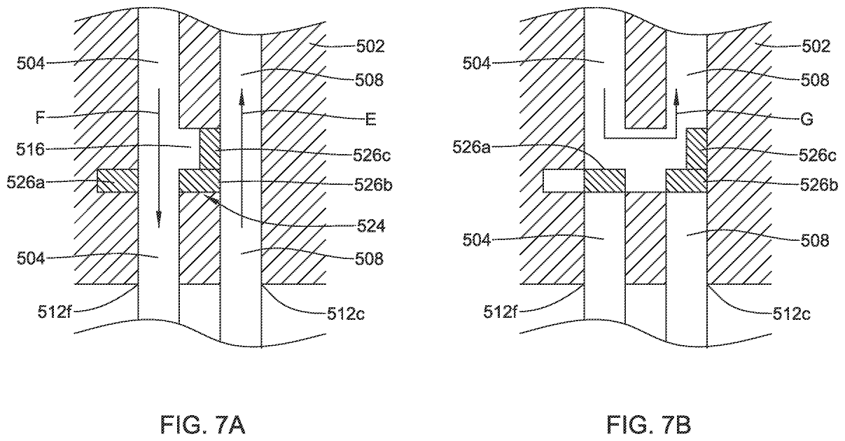

FIG. 7A is a schematic cross-sectional view of another illustrative valve 524 that may be used with the manifold 502 of FIG. 5, in a first configuration, and FIG. 7B is a schematic cross-sectional view of the valve 524 in a second configuration. The valve 524 is positioned at least partially within a connecting channel 516 extending between the gas supply tubing 504 and the water supply tubing 508. The connecting channel 516 fluidly couples the gas supply tubing 504 and the water supply tubing 508. In the illustrative embodiment of FIGS. 7A and 7B, the valve 524 may be a slide valve including a plurality of sliding elements 526a, 526b, 526c (collectively, 526). A first sliding element 526a is positioned adjacent to the gas supply tubing 504 and is configured to slide into and out of the gas supply tubing 504 to selectively block the lumen thereof. A second sliding element 526b is positioned adjacent to the water supply tubing 508 and is configured to slide into and out of the water supply tubing 508 to selectively block the lumen thereof. A third sliding element 526c is positioned adjacent to the connecting channel 516 and is configured to slide out of and into the connecting channel 516 to selectively fluidly connect the gas supply tubing 504 with the water supply tubing 508. The sliding elements 526 are sized and shaped to provide a fluid tight seal within the respective lumen in which they are positioned. However, other valve types and valve configurations may be used, as desired. While not explicitly shown, an actuation mechanism, such as, but not limited to a thumb switch, a slide bar or an actuation tab, may be positioned exterior to the manifold 502 such that the user may selectively move the valve 524 between the first and second configurations.

In the first configuration illustrated in FIG. 7A, the valve 524 is positioned to allow for normal function of the endoscope. For example, when the valve 524 is in the first configuration, the third sliding element 526a is positioned within the connecting channel 516 to fluidly isolate the gas supply tubing 504 and the water supply tubing 508 and the first and second sliding elements 526a, 526b are positioned adjacent to or outside of the lumens of the gas supply tubing 504 and 508 such that the gas inlet 512a, 512e is in fluid communication with the air outlet 512f and the water inlet 512c is in fluid communication the second water outlet 512b, 512d. The manifold 502 directs air or gas from the processing unit 210 (or other external source) into the water reservoir 570, along flow path F. The flow of air/gas can be controlled at the gas/water valve 140, as described above. For example, when the gas/water valve 140 is depressed, the air/gas is received into the manifold 502 at a first inlet or opening 512a, 512e and exits the manifold 502 at an air outlet opening 512f via the gas supply tubing 504. Pressure generated by the air within the sealed water reservoir 570 directs water up the water supply tubing 508. Water enters the manifold at a water inlet 512c via the water supply tubing 508. Depending on whether or not the irrigation pump 315 is active, water travels along flow path E to the lens wash supply line 245a and/or the irrigation supply line 255a.

When the endoscopic procedure is complete and pre-cleaning of the endoscope 100 is to be performed, the valve 524 is actuated to its second configuration, as shown in FIG. 7B. This may be performed by manually sliding an actuation mechanism or by activating an electronically controlled actuator. When the valve 524 is in the second configuration, the gas inlet 512a, 512e is in fluid communication with the water outlet 512b, 512d of the manifold 502 via the connecting channel 516. For example, the sliding elements 526 are laterally translated such that the first sliding element 526a blocks the flow of air/gas within the gas supply tubing 504 and the second sliding element 526b blocks the flow of fluid from the reservoir 570. Further, the third sliding element 526c is moved from laterally displaced from the connecting channel 516 such that the lumen of the gas supply tubing 504 is in fluid communication with the lumen of the water supply tubing 508 via the connecting channel 516. Additionally, in the second configuration the first sliding element 526a prevents air/gas from entering the reservoir 570 and diverts the air/gas into the water supply tubing 508, as shown at flow path G. The air then enters the endoscope and pushes water out of the irrigation and lens wash channels 255a, 245a.

To perform the pre-cleaning procedure, water may first be flushed through the lens wash and irrigation channels 245a, 255a with the valve 524 in the first configuration. The valve 524 may then be actuated to the second configuration and air/gas is directed into the lens wash and irrigation channels 245a, 255a to remove as much of the flushed water from the channels 245a, 255a as possible.

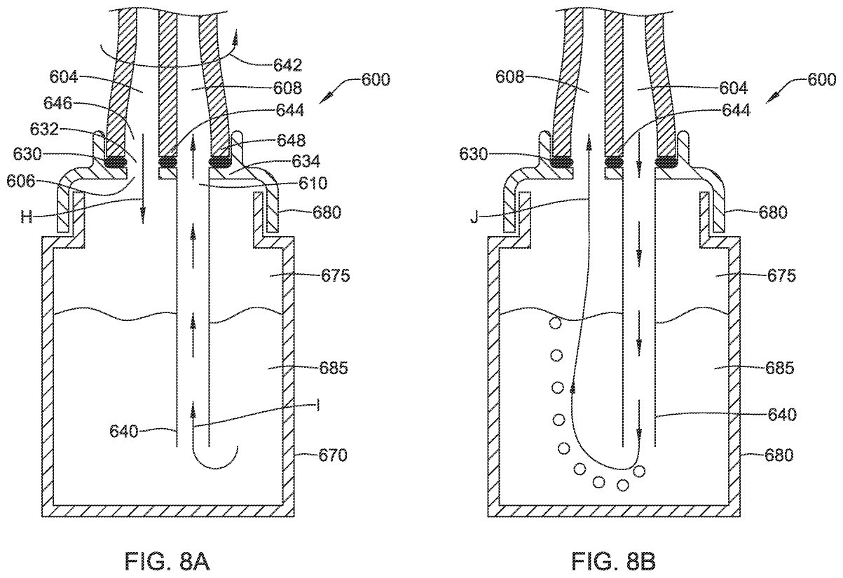

FIG. 8A depicts an illustrative container and tube set or assembly 600 for use with an endoscope system in a first configuration, and FIG. 8B depicts the illustrative container and tube set of FIG. 8A in a second configuration. The container and tube set 600 is arranged and configured for distributing fluid to an endoscope system. The container and tube set or assembly 600 may be further arranged and configured to divert air or CO₂ supplied by the air pump 215 (or other gas source) to the lens wash supply line 245a and/or the irrigation supply line 255a to perform the pre-cleaning process. Generally, the air and lens wash tubes may be rotatably coupled to the cap 680. The system apart from the container and tube set 600 includes components similar to the endoscope and endoscope systems described with regard to FIGS. 1-4; however, not all features may be described or shown here if not pertinent to the fluid circuit of the system.

The fluid reservoir 670 is shown with reservoir top or cap 680, which may be removably attachable to the top portion of the reservoir 670 (e.g., in a bottle and threaded cap arrangement). The cap 680 may be removably attached in order to replenish fluid in the reservoir when it becomes depleted. Alternatively, the reservoir bottom and cap 680 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

The reservoir cap 680 may be connected in fluid communication with a lumen of a gas supply tube 604 and a lumen of a water supply tube 608. In some instances, a tubing manifold similar in form and function to the manifold 502 described with respect to FIG. 5 may be used. In such an instance, the gas supply tube may be a shared gas supply/alternate gas supply tubing 604 and the water supply tube may be a shared lens wash supply/irrigation supply tubing 608. The gas supply tubing 604 extends from a second end external to the reservoir 670 to a first end adjacent to an opening 606 in the cap 680. A lumen extends through the gas supply tubing 604 for receiving a flow of air and/or gas therethrough. The shared water supply tubing 608 extends from a second end external to the reservoir 670 to a first end adjacent a reservoir opening 610 in the cap 680. A lumen extends through the water supply tubing 608 for receiving a flow of fluid therethrough. The second ends of the gas supply tubing 604 and the water supply tubing 608 may be coupled to a manifold (if so provided) or a connector portion 265 of an endoscope system. The first end of the water supply tubing 608 is in fluid communication with a lumen of a water pick-up tube 640. The water pick-up tube 640 may be fixedly secured to or within the reservoir opening 610 in the cap 680. The water pick-up tube 640 extends towards a bottom of the reservoir 670 such that the first end extends into the fluid 685 when present in the reservoir 670. A reservoir gap 675 is between the cap 680 and the fluid 685.

The first ends of the gas supply tubing 604 and the water supply tubing 608 are rotatably coupled to the cap 680. In some embodiments, the first ends of the gas supply tubing 604 and the 608 may be secured to a rotatable member 644 configured to mechanically engage the cap 680. In other embodiments, the first ends of the gas supply tubing 604 and the water supply tubing 608 may form the rotatable member (e.g., a single monolithic structure) and directly mechanically engage the cap 680. Some illustrative mechanical engagements may include, but are not limited to, a threaded arrangement, a snap fit, a friction fit, etc. The mechanical engagement may be configured to prevent the first ends of the gas supply tubing 604 and the water supply tubing 608 from accidentally disengaging from the cap 680 while providing a pressure tight seal and allowing the first ends of the gas supply tubing 604 and the water supply tubing 608 to rotate as shown at arrow 642. The rotatable member 644 includes openings or apertures 646, 648 configured to align with the openings 606, 610 in the cap 680. A gasket or sealing member 630 may be positioned between the first ends of the gas supply tubing 604 and the water supply tubing 608 to provide a pressure tight seal between the tubes 604, 608 and the cap 680. When so provided, the gasket 630 includes openings or apertures 632, 634 configured to align with the openings 606, 610 in the cap 680 and the openings 646, 648 in the rotatable member 644.

In the first configuration, the rotatable member 644 is positioned to allow for normal function of the endoscope. For example, when the rotatable member 644 is in the first configuration, the gas supply tubing 604 aligns with a first opening 606 in the cap 680 and the water supply tubing 608 aligns with a second opening 610 in the cap and is in fluid communication with the water pick-up tube 640. Air or gas is directed from the processing unit 210 (or other external source) into the water reservoir 670, along flow path H. The flow of air/gas can be controlled at the gas/water valve 140, as described above. For example, when the gas/water valve 140 is depressed, the air/gas is received into the reservoir 670. Pressure generated by the air within the sealed water reservoir 670 directs water up the pick-up tube 640 and the water supply tubing 608, along flow path I. Depending on the configuration of the tubing and whether or not the irrigation pump 315 is active, water travels along a flow path (a) to the lens wash supply line 245a or (b) to both the lens wash supply line 245a and the irrigation supply line 255a. When the endoscopic procedure is complete and pre-cleaning of the endoscope is to be performed, the rotatable member 644 (and thus the gas supply tubing 604 and the water supply tubing 608) is actuated to its second configuration, as shown in FIG. 8B. The rotatable member 644 may be rotated about 180° or until the gas supply tubing 604 aligns with the second opening 610 in the cap and is in fluid communication with the water pick-up tube 640 and the water supply tubing 608 aligns with the first opening 606 in the cap 680. In this second configuration, the lumen of the gas supply tubing 604 rotates to become fluidly coupled with the water pickup tube 640, while the lumen of the water supply tubing 608 rotates to become fluidly coupled with the air opening 606 in the cap. In this configuration, air flows from the gas supply tubing 604, through the water pickup tube 640 down to the bottom of the reservoir 670, and exits into the fluid, along flow path J. The air/gas then bubbles up to the top of the reservoir 670 and achieves positive pressure in the reservoir 670 with air from the processor pump or alternative source. When the user opens the valve for lens wash in the scope handle, the air pressure at the top of the bottle then flows through the air opening 606 in the cap 680, which is now fluidically coupled to the water supply tubing 608 in the tube set, along flow path J within the water supply tubing 608, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air moving along flow path J may flush water out of both the lens wash and irrigation channels 245a, 255a. In some examples, this may be performed without activation of the irrigation pump 315.

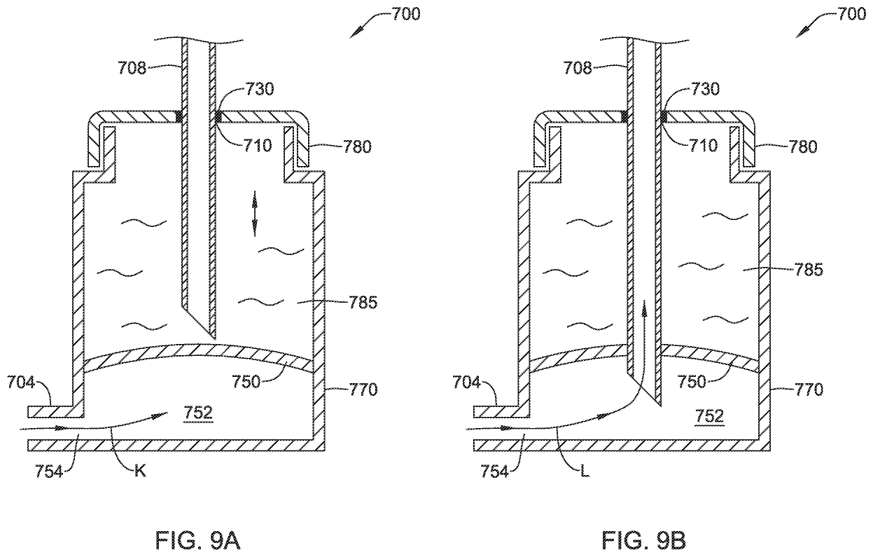

FIG. 9A depicts another illustrative container and tube set or assembly 700 for use with an endoscope system in a first configuration and FIG. 9B depicts the illustrative container and tube set of FIG. 9A in a second configuration. The container and tube set 700 is arranged and configured for distributing fluid to an endoscope system. The container and tube set 700 may be further arranged and configured to divert air or CO₂ supplied by the air pump 215 (or other gas source) to the lens wash supply line 245a and/or the irrigation supply line 255a to perform the pre-cleaning process. The system apart from the container and tube set 700 includes components similar to the endoscope and endoscope systems described with regard to FIGS. 1-4; however, not all features may be described or shown here if not pertinent to the fluid circuit of the system. In the illustrated embodiment, a different means for performing insufflation may be required.

The fluid reservoir 770 is shown with reservoir top or cap 780, which may be removably attachable to the top portion of the reservoir 770 (e.g., in a bottle and threaded cap arrangement). The cap 780 may be removably attached in order to replenish fluid in the reservoir when it becomes depleted. Alternatively, the reservoir bottom and cap 780 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

The reservoir 770 includes an inflatable bladder 750. In some cases, the inflatable bladder 750 may be positioned in a bottom portion (e.g., opposite the cap end) of the reservoir 770, although this is not required. A gas supply tubing 704 extends from a second end external to the reservoir 770 to a first end adjacent an opening 754 in the reservoir 770 such that the gas supply tubing 704 is in fluid communication with an interior or a cavity 752 of the inflatable bladder 750. A lumen extends through the gas supply tubing 704 for receiving a flow of air and/or gas therethrough. In some instances, a tubing manifold similar in form and function to the manifold 502 described with respect to FIG. 5 may be used. In such an instance, the gas supply tube may be a shared gas supply/alternate gas supply tubing 704. The inflatable bladder 750 fluidly isolates the air/gas received from the gas supply tubing 704 from the water 785 in the reservoir 770. The inflatable bladder 750 is configured to expand as air/gas flows into the cavity 752, along flow path K. In the absence of a positive air flow, the inflatable bladder 750 may deflate or contract. Air flow to the interior 752 of the bladder 750 may be controlled to inflate (expand) or deflate (contract) the bladder 750, as desired.

The reservoir cap 780 may be connected in fluid communication with a lumen of a water supply tube 708. In some instances, a tubing manifold similar in form and function to the manifold 502 described with respect to FIG. 5 may be used. In such an instance, the water supply tube 708 may be a shared lens wash supply/irrigation supply tubing 708. A gasket or sealing member 730 may be positioned in the opening 710 to provide a fluid tight seal between the water supply tubing 708 and the cap 780. The shared water supply tubing 708 extends from a second end external to the reservoir 770 to a first end within an intermediate portion of the reservoir 770. The first end of the water supply tubing 708 is in operative fluid communication with an interior of the reservoir 770. A lumen extends through the water supply tubing 708 for receiving a flow of fluid therethrough. The second ends of the gas supply tubing 704 and the water supply tubing 708 may be coupled to a manifold (if so provided) or a connector portion 265 of an endoscope system. The water supply tubing 708 is configured to be axially translated along a longitudinal axis thereof. In some cases, a thumb wheel (not explicitly shown) in contact with the water supply tubing 708 may be used to move the tubing 708 up and down. For example, the first end of the water supply tubing 708 is movable to selectively fluidly couple the water supply tubing 708 with the water 785 in the reservoir 770 or the air/gas in the interior 752 of the inflatable bladder 750. The first end of the water supply tubing 708 may comprise a needle such that the water supply tubing 708 may puncture the bladder 750 as the water supply tubing 708 is axially displaced toward a bottom of the reservoir 770.

In the first configuration (FIG. 9A), the water supply tubing 708 is positioned to allow for normal function of the endoscope. For example, air/gas is received into the interior 752 of the bladder 750. As the pressure increases the bladder expands and translates upward, pushing water up the water supply tubing 708 and to the endoscope. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the water supply tubing 708 is translated towards the bottom of the reservoir 770, as shown in FIG. 9B. The first end of the water supply tubing 708 may puncture or penetrate the bladder 750. It is contemplated that the bladder 750 may be formed from a self-sealing material such that a seal is formed around the water supply tubing 708 and the hole created by the tubing 708 in the bladder 750 is closed when the water supply tubing 708 is translated towards the cap 780. Once the water supply tubing 708 penetrates the bladder 750, as air flows into the interior 752 of the bladder 750 the air/gas travels up the lumen of the water supply tubing 708, along flow path L and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air moving along flow path L may flush water out of both the lens wash and irrigation channels 245a, 255a. In some examples, this may be performed without activation of the irrigation pump 315.

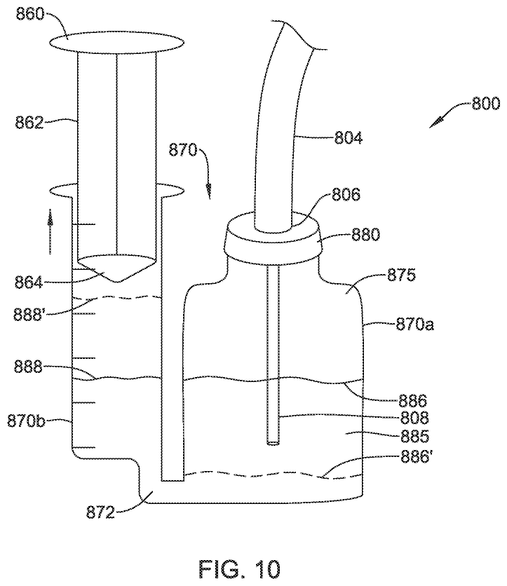

FIG. 10 depicts another illustrative container and tube set or assembly 800 for use with an endoscope system in a first configuration. The container and tube set 800 is arranged and configured for distributing fluid to an endoscope system. The container and tube set 800 may be further arranged and configured to divert air or CO₂ supplied by the air pump 215 (or other gas source) to the lens wash supply line 245a and/or the irrigation supply line 255a to perform the pre-cleaning process. The system apart from the container and tube set 800 includes components similar to the endoscope and endoscope systems described with regard to FIGS. 1-4; however, not all features may be described or shown here if not pertinent to the fluid circuit of the system.

The fluid reservoir 870 is shown with a first chamber 870a having a top portion and a bottom portion and a second chamber 870b having a top portion and a bottom portion. The first and second chambers 870a, 870b may have a same volume or differing volumes. The bottom portion of the second chamber 870b is fluidly coupled to the bottom portion of the first chamber 870a via, for example, a channel or connection tube 872. A reservoir top or cap 880 may be removably attachable to the top portion of the first chamber reservoir 870a (e.g., in a bottle and threaded cap arrangement). The cap 880 may be removably attached in order to replenish fluid in the reservoir 870 when it becomes depleted. Alternatively, the reservoir bottom and cap 880 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

A pressure control device 860 may be disposed in the top portion of the second chamber 870b. In some embodiments, the pressure control device 860 may be a syringe including a plunger 862 and a sealing tip 864. The sealing tip 864 may provide a pressure tight seal between the pressure control device 860 and an inner wall of the second chamber 870b. The pressure control device 860 may be axially displaced within the second chamber 870b using hand pressure. In other embodiments, the pressure control device 860 may be a pneumatic plunger which may be axially displaced within the second chamber 870b using air pressure. In yet another embodiment, the pressure control device 860 may be a solenoid.

The pressure control device 860 is configured to raise and lower a level 886 of fluid 885 in the first chamber 870a. For example, the pressure control device 860 may be axially displaced towards the top portion of the second chamber 870b to draw fluid 885 from the first chamber 870a into the second chamber 870b. This may cause the water in the first chamber to drop to a level 886′ lower than an original level 886 while raising the water in the second chamber 870b to a level 888′ greater than an original level 888. A reservoir gap 875 is between the cap 880 and the fluid 885.

The reservoir cap 880 may be connected in fluid communication with a lumen of a gas supply tube 804 and a lumen of a water supply tube 808. In some instances, a tubing manifold similar in form and function to the manifold 502 described with respect to FIG. 5 may be used. In such an instance, the gas supply tube may be a shared gas supply/alternate gas supply tubing 804 and the water supply tube may be a shared lens wash supply/irrigation supply tubing 808. The gas supply tube 804 and the water supply tube 808 may be coaxially arranged with the water supply tube 808 extending within and through the lumen of the gas supply tube 804 along a portion of the length of the gas supply tube 804. However, this is not required. In some cases, the gas supply tube 804 and the water supply tube 808 may extend side by side. The shared gas supply tubing 804 extends from a second end external to the reservoir 870 to a first end adjacent to an opening 806 in the cap 880. A lumen extends through the gas supply tube 804 for receiving a flow of air and/or gas therethrough. The lumen of the gas supply tube 804 is in fluid communication with the first chamber 870a. The shared water supply tubing 808 extends from a second end external to the reservoir 870 to a first end which extends through the opening 806 and into an interior of the first chamber 870a. A lumen extends through the water supply tube 808 for receiving a flow of fluid therethrough. The second ends of the gas supply tube 804 and the water supply tube 808 may be coupled to a manifold (if so provided) or a connector portion 265 of an endoscope system. The first end of the water supply tube 808 is in selective fluid communication with the bottom portion of the first chamber 870a.

In a first configuration, the water level 886 in the first chamber is maintained to allow for normal function of the endoscope. For example, air/gas is received into the interior of the first chamber 870a. Pressure generated by the air within the sealed water reservoir 870a directs water up the water supply tube 808. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the pressure control device 860 is axially translated within the second chamber 870b to lower a level 886′ of fluid in the first chamber 870a. For example, the seal 864 may be moved towards the top portion of the second chamber 870b to draw fluid from the first chamber 870a into the second chamber 870b. This may raise the fluid level 888′ in the second chamber 870b while lowering the fluid level 886′ in the first chamber 870a. The fluid level 886′ may be lowered until the first end of the water supply tube 808 is in fluid communication with air/gas (or is no longer in fluid communication with the fluid 885). In this second configuration, the first end of the water supply tube 808 is positioned within the air gap 875 such that the lumen of the water supply tube 808 is in fluid communication with air/gas. When the user opens the valve for lens wash in the scope handle, the air pressure at the top of the bottle then flows through the water supply tube 808 in the tube set, and flows through the water lumen into the endoscope, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air may flush water out of both the lens wash and irrigation channels 245a, 255a.

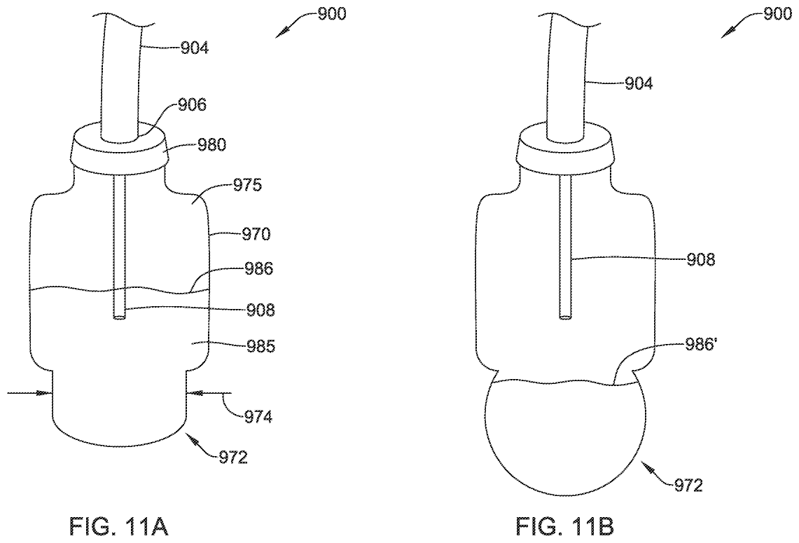

FIG. 11A depicts another illustrative container and tube set or assembly 900 for use with an endoscope system in a first configuration and FIG. 11B depicts the illustrative container and tube set of FIG. 11A in a second configuration. The container and tube set 900 is arranged and configured for distributing fluid to an endoscope system. The container and tube set 900 may be further arranged and configured to divert air or CO₂ supplied by the air pump 215 (or other gas source) to the lens wash supply line 245a and/or the irrigation supply line 255a to perform the pre-cleaning process. The system apart from the container and tube set 900 includes components similar to the endoscope and endoscope systems described with regard to FIGS. 1-4; however, not all features may be described or shown here if not pertinent to the fluid circuit of the system.

The fluid reservoir 970 is shown with a reservoir top or cap 980, which may be removably attachable to the top portion of the reservoir 970 (e.g., in a bottle and threaded cap arrangement). The cap 980 may be removably attached in order to replenish fluid in the reservoir when it becomes depleted. Alternatively, the reservoir bottom and cap 980 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid. A reservoir gap 975 is between the cap 980 and the fluid 985.

The reservoir 970 includes deformable bottom portion 972 opposite the cap 980. The bottom portion 972 may be formed from a same material or a different material as a top portion of the reservoir 970. A biasing force 974 may be applied to the bottom portion 972 to reduce a volume thereof and raise a level 986 of the fluid in the reservoir 970. In some cases, the biasing force 974 may be applied by a person gripping the bottom portion 972. In other examples, the biasing force 974 may be applied by an external device or mechanism. Some illustrative compressive devices may include, but are not limited to, an elastic or rubber band, a two-way zip tie, a single use zip tie, a vice, etc. The compressive devices may be removable to allow the bottom portion 972 to expand and increase in volume (as shown in FIG. 11B). The biasing force may be applied or exerted in raise a fluid level 986 in the reservoir 970 (FIG. 11A) and relaxed or released to lower a fluid level 986′ in the reservoir 970 (FIG. 11B).

The reservoir cap 980 may be connected in fluid communication with a lumen of a gas supply tube 904 and a lumen of a water supply tube 908. In some instances, a tubing manifold similar in form and function to the manifold 502 described with respect to FIG. 5 may be used. In such an instance, the gas supply tube may be a shared gas supply/alternate gas supply tubing 904 and the water supply tube may be a shared lens wash supply/irrigation supply tubing 908. The gas supply tube 904 and the water supply tube 908 may be coaxially arranged with the water supply tube 908 extending within and through the lumen of the gas supply tube 904 along a portion of the length of the gas supply tube 904. Alternatively, the gas supply tube 904 and the water supply tube 908 may extend side by side. The shared gas supply tubing 904 extends from a second end external to the reservoir 970 to a first end adjacent to an opening 906 in the cap 980. A lumen extends through the gas supply tube 904 for receiving a flow of air and/or gas therethrough. The lumen of the gas supply tube 904 is in fluid communication with the interior of the reservoir 970. The shared water supply tubing 908 extends from a second end external to the reservoir 970 to a first end which extends through the opening 906 and into an interior of the reservoir 970. A lumen extends through the water supply tube 908 for receiving a flow of fluid therethrough. The first end of the water supply tube 908 is in selective fluid communication with the fluid 985 in the reservoir 970. The second ends of the gas supply tube 904 and the water supply tube 908 may be coupled to a manifold (if so provided) or a connector portion 265 of an endoscope system.

In the first configuration (FIG. 11A), the water level 986 in the reservoir is maintained to allow for normal function of the endoscope. For example, a compressive or biasing force 974 is placed on the bottom portion 972 of the reservoir 970 such that the water level 986 is above the first end of the water supply tube 908. This selectively fluidly couples the lumen of the water supply tube 908 with the fluid in the reservoir 970. Air/gas is received into the interior of the reservoir 870. Pressure generated by the air within the sealed water reservoir 870 directs water up the water supply tube 908. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the biasing or compressive force 974 on the bottom portion 972 of the reservoir 970 is released, as shown in FIG. 11B. Releasing the biasing force may include a complete removal of the biasing force 974 or a reduction in the force applied. Releasing the biasing force may increase the volume of the bottom portion 972 of the reservoir 970 which lowers the fluid level 986′ in the reservoir. The fluid level 986′ may be lowered until the first end of the water supply tube 908 is in fluid communication with air/gas (or is no longer in fluid communication with the fluid 985). In this second configuration, the first end of the water supply tube 908 is positioned within the air gap 975 such that the lumen of the water supply tube 908 is in fluid communication with air/gas. When the user opens the valve for lens wash in the scope handle, the air pressure at the top of the bottle then flows through the water supply tube 908 in the tube set, and flows through the water lumen into the endoscope, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air may flush water out of both the lens wash and irrigation channels 245a, 255a.

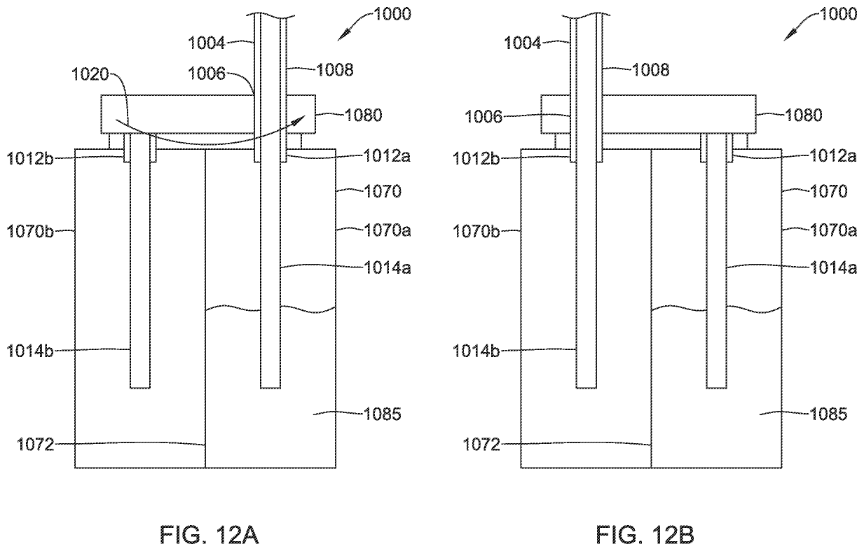

FIG. 12A depicts another illustrative container and tube set or assembly 1000 for use with an endoscope system in a first configuration, and FIG. 12B depicts the illustrative container and tube set of FIG. 12A in a second configuration. The container and tube set 1000 is arranged and configured for distributing fluid to an endoscope system. The container and tube set 1000 may be further arranged and configured to divert air or CO₂ supplied by the air pump 215 (or other gas source) to the lens wash supply line 245a and/or the irrigation supply line 255a to perform the pre-cleaning process. The system apart from the container and tube set 1000 includes components similar to the endoscope and endoscope systems described with regard to FIGS. 1-4; however, not all features may be described or shown here if not pertinent to the fluid circuit of the system.

The fluid reservoir 1070 is shown with a first chamber 1070a having a top portion and a bottom portion and a second chamber 1070b having a top portion and a bottom portion. In some cases, the fluid reservoir 1070 may include more than two chambers. The chambers 1070a, 1070b may have a similar volume or differing volumes, as desired. In some embodiments, the first chamber 1070a may be configured to receive a fluid 1085 while the second chamber 1070b is filled with air. The first and second chambers 1070a, 1070b may be positioned side by side or may formed as a single monolithic structure with a dividing wall 1072. The first and second chambers 1070a, 1070b may be fluidly coupled adjacent a top portion thereof or may be fluidly isolated, as desired. A reservoir top or cap 1080 may be removably attachable to the top portion of the first and second chamber reservoirs 1070a, 1070b (e.g., in a bottle and threaded cap arrangement). The cap 1080 may be removably attached in order to replenish fluid in the reservoir when it becomes depleted. Alternatively, the reservoir bottom and cap 1080 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

Each chamber 1070a, 1070b may include its own dedicated set of air tubes 1012a, 1012a (collectively, 1012) and water supply tubes 1014a, 1014b (collectively, 1014). For example, the first chamber 1070a may include an air tube 1012a and a water supply tube 1014a. The air tube 1012a and the water supply tube 1014a may be coaxially arranged with the water supply tube 1014a extending within and through the lumen of the gas tube 1012a along a portion of the length of the gas tube 1012a. Similarly, the second chamber 1070b may include an air tube 1012b and a water supply tube 1014b. The air tube 1012b and the water supply tube 1014b may be coaxially arranged with the water supply tube 1014b extending within and through the lumen of the gas tube 1012b along a portion of the length of the gas tube 1012b. The air tubes 1012 and the water supply tubes 1014 may be secured to their respective chambers 1070a, 1070b with an insert, gasket, etc. configured to maintain the tubes 1012, 1014 in a desired position.

The reservoir cap 1080 may be connected in fluid communication with a lumen of a gas supply tube 1004 and a lumen of a water supply tube 1008. In some instances, a tubing manifold similar in form and function to the manifold 502 described with respect to FIG. 5 may be used. In such an instance, the gas supply tube may be a shared gas supply/alternate gas supply tubing 1004 and the water supply tube may be a shared lens wash supply/irrigation supply tubing 1008. The gas supply tube 1004 and the water supply tube 1008 may be coaxially arranged with the water supply tube 1008 extending within and through the lumen of the gas supply tube 1004 along a portion of the length of the gas supply tube 1004. In other embodiments, the gas supply tube 1004 and the water supply tube 1008 may extend side by side. The shared gas supply tubing 1004 extends from a second end external to the reservoir 1070 to a first end adjacent to an opening 1006 in the cap 1080. A lumen extends through the gas supply tube 1004 for receiving a flow of air and/or gas therethrough. The lumen of the gas supply tube 1004 is in selective fluid communication with either of the air tubes 1012a, 1012b of the chamber 1070a, 1070b (e.g., one at a time). The shared water supply tubing 1008 extends from a second end external to the reservoir 1070 to a first end adjacent to an opening 1006 in the cap 1080. A lumen extends through the water supply tube 1008 for receiving a flow of fluid therethrough. The lumen of the water supply tube 1008 is in selective fluid communication with either of the water supply tubes 1012a, 1012b of the chambers 1070a, 1070b (e.g., one at a time. The second ends of the gas supply tube 1004 and the water supply tube 1008 may be coupled to a manifold (if so provided) or a connector portion 265 of an endoscope system.

The reservoir cap 1080 may be rotated, as shown at arrow 1020, to selective couple the gas supply tube 1004 and the water supply tube 1008 with either the first set of gas and water supply tubes 1012a, 1014a or the second set of gas and water supply tubes 1012b, 1014b. The reservoir cap 1080 and/or the reservoir 1070 may include mechanisms to seal the gas supply tube 1004 and the water supply tube 1008 with the tubes 1012, 1014, including, but not limited to o-rings, spring-loaded fits, etc. In a first configuration (FIG. 12A), the reservoir cap 1080 is positioned to allow for normal function of the endoscope. For example, when the reservoir cap 1080 is in the first configuration, the gas supply tube 1004 and the water supply tube 1008 align with the first set of gas and water supply tubes 1012a, 1014a. Thus, the water supply tube 1008 is in fluid communication with the fluid or water 1085 inside the first chamber 1070a. Air or gas is directed from the processing unit 210 (or other external source) into the first chamber 1070a of the water reservoir 1070. The flow of air/gas can be controlled at the gas/water valve 140, as described above. For example, when the gas/water valve 140 is depressed, the air/gas is received into at least the first chamber 1070a. Pressure generated by the air within the sealed water reservoir 1070 directs water up the water supply tube 1014a and the water supply tube 1008. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the cap 1080 (and thus the gas supply tube 1004 and the water supply tube 1008) is actuated to its second configuration, as shown in FIG. 12B. The cap 1080 may be rotated about 180° or until the gas supply tube 1004 and water supply tube 1008 align with the second set of gas and water supply tubes 1012b, 1014b. In this second configuration, the lumen of the water supply tube 1008 rotates to become fluidly coupled with the water supply tube 1014b of the second chamber 1070b. In this configuration, air flows through the gas supply tube 1004 and the gas tube 1012b into at least the second chamber 1070b. The water supply tube 1008 is in fluid communication with the second water supply tube 1014b which in turn is in fluid communication with the air/gas in the second chamber 1070b. When the user opens the valve for lens wash in the scope handle, the air pressure within the second chamber 1070b flows through the gas supply tube 1004 and the water supply tube 1014b in the tube set, and flows through the water lumen into the endoscope, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air may flush water out of both the lens wash and irrigation channels 245a, 255a.

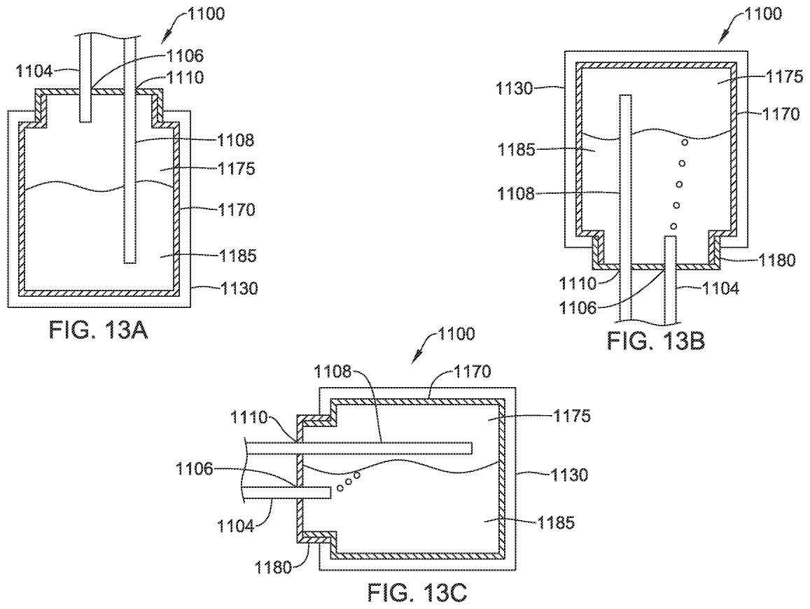

FIG. 13A depicts another illustrative container and tube set or assembly 1100 for use with an endoscope system in a first configuration, FIG. 13B depicts the illustrative container and tube set of FIG. 13A in a second configuration, and FIG. 13C depicts the illustrative container and tube set of FIG. 13A in an alternative second configuration. The container and tube set 1100 is arranged and configured for distributing fluid to an endoscope system. The container and tube set 1100 may be further arranged and configured to divert air or CO₂ supplied by the air pump 215 (or other gas source) to the lens wash supply line 245a and/or the irrigation supply line 255a to perform the pre-cleaning process. The system apart from the container and tube set 1100 includes components similar to the endoscope and endoscope systems described with regard to FIGS. 1-4; however, not all features may be described or shown here if not pertinent to the fluid circuit of the system.

The fluid reservoir 1170 is shown with reservoir top or cap 1180, which may be removably attachable to the top portion of the reservoir 1170 (e.g., in a bottle and threaded cap arrangement). The cap 1180 may be removably attached in order to replenish fluid in the reservoir when it becomes depleted. Alternatively, the reservoir 1170 and reservoir top 1180 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

The reservoir cap 1180 may be connected in fluid communication with a tubing manifold or a connecting portion 265 via a portion of shared gas supply/alternate gas supply tubing 1104. The shared tubing 1104 extends from a second end external to the reservoir 1170 through a reservoir opening 1106 in the reservoir top 1180, terminating at a first end within the reservoir gap 1175, at or below the top opening 1106, but not extending into the remaining fluid 1185 in the reservoir 1170. A lumen extends through the gas supply tube 1104 for receiving a flow of air and/or gas therethrough. The reservoir top 1180 may also be connected in fluid communication with tubing manifold or a connecting portion 265 via a portion of a shared lens wash supply/irrigation supply tubing 1108. The water supply tubing 1108 extends from a second end at an opening of manifold 502 through a reservoir opening 1110 in the top 1180, terminating in a first end within the remaining fluid 1185 at or substantially at the bottom of reservoir 1170. A lumen extends through the water supply tube 1108 for receiving a flow of fluid therethrough. In some embodiments, the gas supply tubing 1104 and the water supply tubing 1108 may enter the top 1180 through a single or common opening. For example, the gas supply tubing 1104 and the water supply tubing 1108 may be coaxially arranged.

In a first configuration (FIG. 13A), air or gas is directed from the processing unit 210 (or other external source) into the water reservoir 1170. The flow of air/gas can be controlled at the gas/water valve 140, as described above. For example, when the gas/water valve 140 is depressed, the air/gas is received into the reservoir 1170. Pressure generated by the air within the sealed water reservoir 1170 directs water up the water supply tube 1108. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the reservoir 1170 may be rotated or inverted such that the fluid 1185 is adjacent to the cap 1180, as shown in FIG. 13B. Alternatively, the reservoir 1170 may be rotated 90° so that the reservoir 1170 is generally orthogonal to the configuration of FIG. 13A with the water supply tube 1108 positioned above the gas supply tube 1104, as shown in FIG. 13C. A rotatable bottle cage 1130 may be provided about the reservoir 1170 to lock the reservoir in a desired configuration. In the second configurations of FIGS. 13B or 13C, the lumen of the gas supply tube 1104 is in fluid communication with the water or fluid 1185 and while the lumen of the water supply tube 1108 rotates to become fluidically coupled with the air gap 1175. In this configuration, air flows through the gas supply tube 1104 and into the water 1185. It then bubbles up to the top of the reservoir 1170, achieving positive pressure in the reservoir 1170 with air from the processor pump or CO₂ source. When the user opens the valve for lens wash in the scope handle, the air pressure at the top of the bottle then flows through the water supply tube 1108, and flows through the water lumen into the endoscope, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air may flush water out of both the lens wash and irrigation channels 245a, 255a.

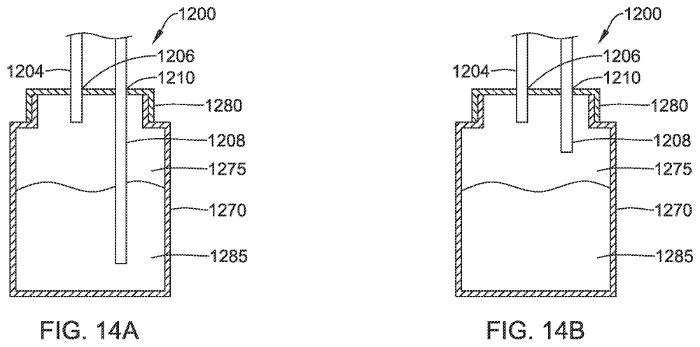

FIG. 14A depicts another illustrative container and tube set or assembly 1200 for use with an endoscope system in a first configuration and FIG. 14B depicts the illustrative container and tube set of FIG. 14A in a second configuration. The container and tube set 1200 is arranged and configured for distributing fluid to an endoscope system. The container and tube set 1200 may be further arranged and configured to divert air or CO₂ supplied by the air pump 215 (or other gas source) to the lens wash supply line 245a and/or the irrigation supply line 255a to perform the pre-cleaning process. The system apart from the container and tube set 1200 includes components similar to the endoscope and endoscope systems described with regard to FIGS. 1-4; however, not all features may be described or shown here if not pertinent to the fluid circuit of the system.

The fluid reservoir 1270 is shown with reservoir top or cap 1280, which may be removably attachable to the top portion of the reservoir 1270 (e.g., in a bottle and threaded cap arrangement). The cap 1280 may be removably attached in order to replenish fluid in the reservoir when it becomes depleted. Alternatively, the reservoir bottom and top 1280 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

The reservoir cap 1280 may be connected in fluid communication with a tubing manifold or a connecting portion 265 via a portion of shared gas supply/alternate gas supply tubing 1204. The shared tubing 1204 extends from a second end external to the reservoir 1270 through a reservoir opening 1206 in the reservoir top 1280, terminating at a first end within the reservoir gap 1275, at or below the top opening 1206, but not extending into the remaining fluid 1285 in the reservoir 1270. A lumen extends through the gas supply tube 1204 for receiving a flow of air and/or gas therethrough. The reservoir top 1280 may also be connected in fluid communication with tubing manifold or a connecting portion 265 via a portion of a shared lens wash supply/irrigation supply tubing 1208. The water supply tubing 1208 extends from a second end at an opening of manifold 502 through a reservoir opening 1210 in the top 1280, terminating in a first end within the remaining fluid 1285 at or substantially at the bottom of reservoir 1270. The water supply tubing 1208 is configured to be axially translated along a longitudinal axis thereof. In some cases, a thumb wheel (not explicitly shown) in contact with the water supply tubing 1208 may be used to move the tubing 1208 up and down. For example, the first end of the water supply tubing 1208 is movable to selectively fluidly couple the water supply tubing 1208 with the water 1285 in the reservoir 1270 or the air/gas in the air gap 1275. A lumen extends through the water supply tube 1208 for receiving a flow of fluid therethrough. In some embodiments, the gas supply tubing 1204 and the water supply tubing 1208 may enter the top 1280 through a single or common opening. For example, the gas supply tubing 1204 and the water supply tubing 1208 may be coaxially arranged.

In a first configuration (FIG. 14A), air or gas is directed from the processing unit 210 (or other external source) into the water reservoir 1270. The flow of air/gas can be controlled at the gas/water valve 140, as described above. For example, when the gas/water valve 140 is depressed, the air/gas is received into the reservoir 1270. Pressure generated by the air within the sealed water reservoir 1270 directs water up the water supply tube 1208. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the water supply tubing 1208 is translated towards the cap 1280 of the reservoir 1270, as shown in FIG. 14B. In the second configuration of FIG. 14B, the lumen of the water supply tube 1208 is axially translated to become fluidically coupled with the air gap 1275. In this configuration, air flows through the gas supply tube 1204 and into the reservoir 1270 achieving positive pressure in the reservoir 1270 with air from the processor pump or CO2 source. When the user opens the valve for lens wash in the scope handle, the air pressure at the top of the bottle then flows through the water supply tube 1208, and flows through the water lumen into the endoscope, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air may flush water out of both the lens wash and irrigation channels 245a, 255a.

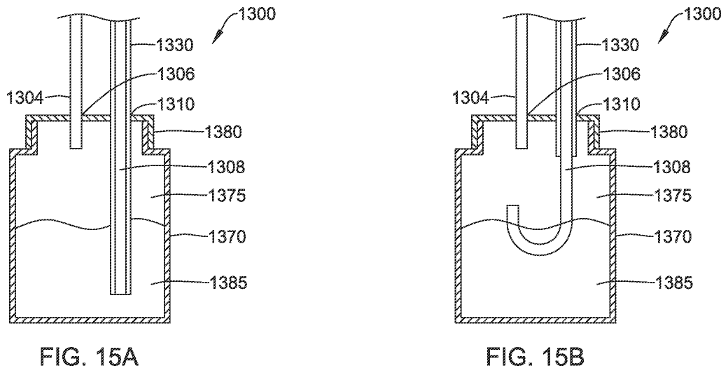

FIG. 15A depicts another illustrative container and tube set or assembly 1300 for use with an endoscope system in a first configuration and FIG. 15B depicts the illustrative container and tube set of FIG. 15A in a second configuration. The container and tube set 1300 is arranged and configured for distributing fluid to an endoscope system. The container and tube set 1300 may be further arranged and configured to divert air or CO₂ supplied by the air pump 215 (or other gas source) to the lens wash supply line 245a and/or the irrigation supply line 255a to perform the pre-cleaning process. The system apart from the container and tube set 1300 includes components similar to the endoscope and endoscope systems described with regard to FIGS. 1-4; however, not all features may be described or shown here if not pertinent to the fluid circuit of the system.

The fluid reservoir 1370 is shown with reservoir top or cap 1380, which may be removably attachable to the top portion of the reservoir 1370 (e.g., in a bottle and threaded cap arrangement). The cap 1380 may be removably attached in order to replenish fluid in the reservoir when it becomes depleted. Alternatively, the reservoir bottom and top 1380 may be sealed to each other or they may be manufactured as a single, integral body (e.g., similar to a sealed monolithic rigid or semi-rigid bottle or softer IV bag or pouch). In such embodiments, a fill port may be included on another part of the reservoir for replenishing fluid.

The reservoir cap 1380 may be connected in fluid communication with a tubing manifold or a connecting portion 265 via a portion of shared gas supply/alternate gas supply tubing 1304. The shared tubing 1304 extends from a second end external to the reservoir 1370 through a reservoir opening 1306 in the reservoir top 1380, terminating at a first end within the reservoir gap 1375, at or below the top opening 1306, but not extending into the remaining fluid 1385 in the reservoir 1370. A lumen extends through the gas supply tube 1304 for receiving a flow of air and/or gas therethrough. The reservoir top 1380 may also be connected in fluid communication with tubing manifold or a connecting portion 265 via a portion of a shared lens wash supply/irrigation supply tubing 1308.

The water supply tubing 1308 extends from a second end at an opening of manifold 502 through a reservoir opening 1310 in the top 1380, terminating in a first end within the remaining fluid 1385 at or substantially at the bottom of reservoir 1370. The water supply tubing 1308 may be formed from a shape memory material. The water supply tubing 1308 may have a curved memory set to the first end. The first end may be biased into a generally linear configuration with a tubular shaft 1330 extending over the water supply tubing 1308. The tubular shaft 1330 may have sufficient hoop strength to maintain the water supply tubing in the generally straightened or linear configuration (FIG. 15A). The tubular shaft 1330 is configured to be axially translated along a longitudinal axis thereof. In some cases, a thumb wheel (not explicitly shown) in contact with the water tubular shaft 1330 may be used to move the tubular shaft 1330 up and down. For example, tubular shaft 1330 is movable to remove the biasing force on the water supply tubing 1308 to allow the first end to assume its unbiased memory set curved configuration (FIG. 15B). A lumen extends through the water supply tube 1308 for receiving a flow of fluid therethrough. In some embodiments, the gas supply tubing 1304 and the water supply tubing 1308 may enter the top 1380 through a single or common opening. For example, the gas supply tubing 1304 and the water supply tubing 1308 may be coaxially arranged.

In a first configuration (FIG. 15A), air or gas is directed from the processing unit 210 (or other external source) into the water reservoir 1370. The flow of air/gas can be controlled at the gas/water valve 150, as described above. For example, when the gas/water valve 150 is depressed, the air/gas is received into the reservoir 1370. Pressure generated by the air within the sealed water reservoir 1370 directs water up the water supply tube 1308. Depending on the configuration of the tubing and whether or not the irrigation pump 315 is active, water travels to the lens wash supply line 245a and/or the irrigation supply line 255a.

When the endoscopic procedure is complete and pre-cleaning of the endoscope is to be performed, the tubular shaft 1330 is translated towards the cap 1380 of the reservoir 1370, as shown in FIG. 15B and the first end of the water supply tubing 1308 assumes its unbiased memory set curved configuration. In the second configuration of FIG. 15B, the lumen of the water supply tube 1308 is fluidically coupled with the air gap 1375. In this configuration, air flows through the gas supply tube 1304 and into the reservoir 1370 achieving positive pressure in the reservoir 1370 with air from the processor pump or CO2 source. When the user opens the valve for lens wash in the scope handle, the air pressure at the top of the bottle then flows through the water supply tube 1308, and flows through the water lumen into the endoscope, and through the lens wash tubing within the endoscope, thus flushing the water out of the endoscope. When the lens wash supply tubing 245c and the irrigation supply tubing 255c are fluidly coupled (e.g., via a manifold similar in form and function to manifold 502), the air may flush water out of both the lens wash and irrigation channels 245a, 255a.

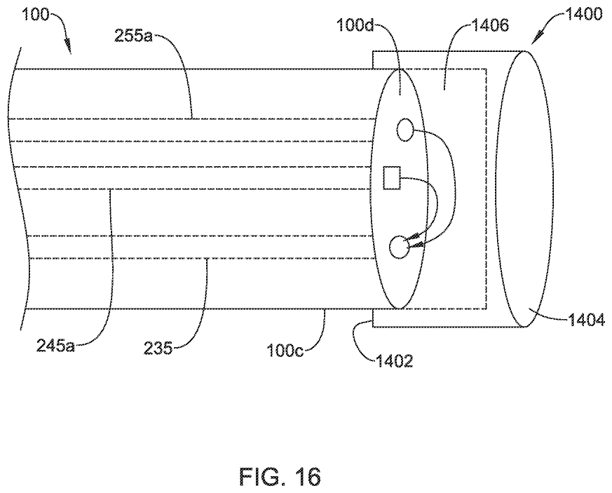

FIG. 16 depicts an illustrative suction cap 1400 for use with an endoscope 100. The suction cap 1400 may have a generally cylindrical shape extending from a first open end 1402 to a second closed end 1404. The suction cap 1400 may further define a cavity 1406 extending from the open first end 1402 towards the closed second end 1404. The cavity 1406 has a length that is sufficient to space the closed second end 1404 from the distal end face 100d when the suction cap 1400 is secured to the distal tip 100c of the endoscope 100. The suction cap 1400 may be sized such that an inner diameter of the cavity 1406 is approximately equal to the outer diameter of the distal tip 100c. The suction cap 1400 may be releasably secured to the distal tip 100c to create a fluid-tight seal using any of a number of connection mechanisms, including, but not limited to, a friction fit, a snap fit, a threaded arrangement, etc.

When the endoscope procedure is complete, the suction cap 1400 is placed on the distal tip 100c to seal the distal tip 100c from atmosphere with the irrigation supply line 255a, lens was supply line 245a, and the working channel 235 in fluid communication with the cavity 1406 of the cap 1400. In some cases, the lens wash tube 245c and the irrigation tube 255c may be uncoupled from the connector portion 265. When so provided, this may be performed by removing the manifold (e.g., manifold 502) from the endoscope 100. The suction valve 145 may then be activated to pull a vacuum. Since the distal tip 100c of the endoscope 100 is capped off, the only places from which fluids can be pulled are the irrigation and lens wash supply lines 255a, 245a. Since the lens wash and irrigation channels 245b, 255b at the proximal end of the umbilical 260 are open to atmosphere, as suction occurs, the water will be distally drawn up through the lens wash supply line 245a and the irrigation supply line 225a to the distal tip 110c of the endoscope 100 and proximally down through the working channel 235, filling the lens wash and irrigation supply lines 245a, 255a with air. This may pull the water out of the lens wash and irrigation supply lines 245a, 255a using a vacuum to pre-clean the endoscope 100.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a container configured to contain a fluid, the container having a bottom portion and a top portion;
a water supply tube including a first end, a second end, and a first lumen extending therethrough, wherein the first lumen is in selective fluid communication with the bottom portion of the container and the second end of the water supply tube is positioned external to the container;
a gas supply tube including a first end, a second end, and a second lumen extending therethrough, wherein the second lumen is in operative fluid communication with the top portion of the container and the second end of the gas supply tube is positioned external to the container;
a manifold including a first inlet for receiving air, a second inlet for receiving fluid from the container, a first air outlet, and a second fluid outlet, wherein the first air outlet is fluidly coupled to the second end of the gas supply tube and the second inlet is fluidly coupled to the second end of the water supply tube; and
a valve positioned within the manifold, the valve movable between a first configuration and a second configuration to control an air flow path,
wherein when the valve is in the first configuration, the first inlet is in fluid communication with the first air outlet, and the second inlet is in fluid communication the second fluid outlet; and
wherein when the valve is in the second configuration, the first inlet is in fluid communication with the second fluid outlet.

2. The container and tube set of claim 1, wherein when the valve is in the first configuration air is configured to flow from the first inlet to the first air outlet and into the container and fluid is configured to flow from the container into the second inlet and to the second fluid outlet.

3. The container and tube set of claim 1, wherein when the valve is in the second configuration air is configured to flow from the first inlet to the second fluid outlet.

4. The container and tube set of claim 1, wherein the second fluid outlet is configured to be in fluid communication with one or more fluid channels of an endoscope.

5. The container and tube set of claim 1, further comprising a connecting channel extending between the gas supply tube and the water supply tube.

6. The container and tube set of claim 5, wherein the valve is at least partially positioned in the connecting channel.

7. The container and tube set of claim 1, wherein the valve comprises a rotating disc.

8. The container and tube set of claim 7, wherein in the first configuration, the rotating disc is configured to fluidly isolate the gas supply tube and the water supply tube.

9. The container and tube set of claim 7, wherein in the second configuration, the rotating disc is configured to block a flow of gas from the gas supply tube to the container and to block a flow of fluid from the container.

10. The container and tube set of claim 1, wherein the valve comprises a slide valve.

11. The container and tube set of claim 10, wherein the slide valve comprises a plurality of slide elements.

12. The container and tube set of claim 10, wherein the slide valve comprises a first sliding element configured to selectively block a flow of gas from the gas supply tube to the container, a second sliding element configured to selectively block a flow of fluid from the container, and a third sliding element configured to selectively fluidly isolate the gas supply tube and the water supply tube.

13. The container and tube set of claim 12, wherein in the first configuration, the third sliding element is disposed within a connecting channel extending between the gas supply tube and the water supply tube.

14. The container and tube set of claim 12, wherein in the second configuration, the first sliding element is disposed within the gas supply tube and the second sliding element is disposed within the water supply tube.

15. A container and tube set arranged and configured to couple to an endoscope for use in an endoscopic procedure, the container and tube set comprising:
a container configured to contain a fluid, the container having a bottom portion and a top portion;
a manifold;
a lens wash supply tube including a first end coupled to the manifold, a second end, and a first lumen extending therethrough, wherein the first lumen is in fluid communication with the manifold and the second end of the lens wash supply tube is configured for connection to an endoscopic system;
a first gas supply tube including a first end coupled to the manifold, a second end, and a second lumen extending therethrough, wherein the second lumen is in fluid communication with the manifold and the second end of the gas supply tube is configured for connection to an endoscopic system;
a water supply tube including a first end, a second end coupled to the manifold, and a third lumen extending therethrough, wherein the third lumen is in fluid communication with the manifold and with the bottom portion of the container;
a second gas supply tube including a first end, a second coupled to the manifold, and a fourth lumen extending therethrough, wherein the fourth lumen is in fluid communication with the manifold and with the top portion of the container; and
a valve positioned within the manifold, the valve movable between a first configuration and a second configuration;
wherein when the valve is in the first configuration, the first lumen of the lens wash supply tube is in fluid communication the third lumen of the water pickup tube, and the second lumen of the first gas supply tube is in fluid communication with the fourth lumen of the second gas supply tube; and
wherein when the valve is in the second configuration, the first lumen of the lens wash supply tube is in fluid communication with the second lumen of the first gas supply tube.

16. The container and tube set of claim 15, further comprising a connecting channel extending between the second gas supply tube and the water supply tube.

17. The container and tube set of claim 16, wherein the valve is at least partially positioned in the connecting channel.

18. The container and tube set of claim 15, wherein the valve comprises a rotating disc.

19. The container and tube set of claim 15, wherein the valve comprises a slide valve.

20. A method of pre-cleaning an endoscope system, the method comprising:
flushing a fluid down a lens wash channel and an irrigation channel;
actuating a valve to fluidly couple a gas supply line with a water supply line; and
initiating a flow of gas through the lens wash channel and the irrigation channel.
